(19) United States
(12) Patent Application Publication
(10) Pub. No.: US 2023/0127198 A1
(43) Pub. Date: Apr. 27, 2023

(54) COMPOSITIONS AND METHODS FOR THE TREATMENT OF TOXIC GAS EXPOSURE

(71) Applicant: THE UAB RESEARCH FOUNDATION, Birmingham, AL (US)

(72) Inventor: Saurabh Aggarwal, Vestavia Hills, AL (US)

(21) Appl. No.: 17/969,836

(22) Filed: Oct. 20, 2022

Related U.S. Application Data
(60) Provisional application No. 63/270,714, filed on Oct. 22, 2021.

(51) Int. Cl. A61K 31/395; A61K 47/18; A61K 31/7048; A61K 9/00; A61P 11/00

(52) U.S. Cl. CPC A61K 31/395; A61K 47/18; A61K 31/7048; A61K 9/0019; A61K 9/0073; A61P 11/00

(57) ABSTRACT

In one aspect, the disclosure relates to a method for treating or preventing at least one symptom of exposure to a toxic gas such as chlorine, bromine, or phosgene in a subject, the method including administering a composition containing AMD3100 or a pharmaceutically acceptable salt thereof to the subject. In one aspect, the composition can be administered by intramuscular injection, intranasally, or by inhalation in an amount of from about 0.01 to about 0.25 mg of AMD3100 per kg of subject body weight.

### CROSS REFERENCE TO RELATED APPLICATIONS

This application claims the benefit of U.S. Provisional Application No. 63/270,714, filed on Oct. 22, 2021, which is incorporated herein by reference in its entirety.

### BACKGROUND

Chlorine (Cl₂) gas is the most common inhalational irritant in the United States and exposure can result in serious adverse effects including lung injury and death. However, currently no specific therapeutic agents are approved by the US Food and Drug Administration (FDA) that can be administered immediately after Cl₂ exposure to minimize lung inflammation and mortality.

Furthermore, current treatment strategies are insufficient and merely palliative. Acute and chronic lung injury post Cl₂ exposure is due to an uncontrolled activation of leukocytes—alveolar macrophages and sequestered neutrophils—in the lung. Excessive recruitment of leukocytes is critical to the pathogenesis of lung injury, and the magnitude and duration of the inflammatory process may ultimately determine the outcome in patients. The binding of the chemokine ligand stromal-derived-factor-1 (SDF-1) to the C-X-C chemokine receptor type 4 (CXCR4) on lung immune, epithelial, and endothelial cells promotes the migration of leukocytes from the circulation to lungs. The SDF-1/CXCR4 axis also propagates the activation and survival of leukocytes in the lungs. Furthermore, both SDF-1 and CXCR4 are elevated in the lungs of Cl₂ exposed animals.

The recruitment of leukocytes to the sites of inflammation and leukocyte-derived inflammatory mediators contribute to the development of tissue injury associated with inflammation. The first step in the pathogenesis of inflammation is transmigration of circulating leukocytes through the activated vascular endothelial cell in the inflamed tissue. During this process, leukocytes are activated to secrete a variety of substances such as growth factors, chemokines and cytokines, proteases, nitric oxide, and reactive oxygen metabolites, which are considered to be one of the primary sources of the tissue injury. Prevention or reduction of leukocyte migration can profoundly attenuate the parenchymal cell dysfunction during inflammation. The accumulated evidence suggests that CXC chemokine CXCL12/stromal cell-derived factor-1 (SDF-1), contributes to the control of the leukocyte life cycle through the activation of CXCR4. Under homeostatic conditions, SDF-1 and its cognate receptor CXCR4 is critical for hemopoietic development and homing of neutrophils and lymphocytes in bone marrow. However, under conditions of stress, elevated SDF-1/CXCR4 signaling in injured tissue promotes the release of neutrophils from bone marrow into blood and the subsequent migration and homing of the circulating neutrophils to the site of injury. Furthermore, ex vivo studies have revealed that SDF-1 acts not only as a chemoattractant, but also as a suppressor of cell death for the lung neutrophils expressing CXCR4. The SDF-1/CXCR4 axis has also been demonstrated to be responsible for the tissue migration of neutrophils and promoting the inflammation in animal models of arthritis and peritonitis.

The role for SDF-1 in lung inflammation has been suggested by animal models of asthma in which a CXCR4 blockade with AMD3100, a CXCR4 antagonist, attenuated both the lymphocyte and eosinophil responses and reduced airway hyperreactivity. SDF-1 was also up-regulated in an animal model of lung fibrosis. The administration of AMD3100 significantly attenuated fibrotic changes in mouse model of bleomycin-induced fibrosis probably by inhibiting the fibrocyte mobilization to the injured lung. Reports have also described the role of SDF-1 in the homing of both malignant metastases and adult stem cells to the lung in mouse models of metastatic cancers. AMD3100 is formally approved by the FDA to reduce metastasis in various malignant diseases. The role of SDF-1/CXCR4 has also been evaluated in lipopolysaccharide (LPS)-induced lung injury, where both SDF-1 levels and surface CXCR4 protein on accumulated neutrophils was increased; furthermore, the in vivo administration of an anti-SDF-1 blocking antibody suppressed airspace neutrophilia in the lungs. However, although the lung is a common site of injury post Cl₂ exposure, the role of SDF-1/CXCR4 in Cl₂-induced lung morbidity and mortality has not yet been determined.

SDF-1 is an 8-kDa small chemotactic cytokine that is often induced by pro-inflammatory stimuli such as TNF-α and IL-1. Interestingly, both IL-1 and TNF-α were elevated post Cl₂ exposure. Furthermore, it has been shown that Cl₂ exposure caused hemolysis and increased plasma levels of cell-free heme, which upregulates inflammatory cytokines such as IL-1 and TNF-1α. In addition, conditions such as hypoxia and growth arrest are able to induce SDF-1 expression, where hypoxia-inducible factor-1 (HIF-1) upregulates the production of SDF-1 by endothelial cells, resulting in increased attraction of progenitor cells. Upregulation of SDF-1 by hypoxia also occurs during cancer development to promote angiogenesis, as has been demonstrated for ovarian cancer. Hypoxia has also been shown to induce CXCR4 expression. SDF-1 is the only chemokine ligand for CXCR4, a unique feature in the otherwise promiscuous chemokine-receptor relationships. Due to its role in angiogenesis in cancer, the SDF-1/CXCR4 axis is therefore approved as a therapeutic target of AMD3100 to arrest cancer metastasis. Functional characterization of the CXCR4 promoter has revealed that Nuclear Respiratory Factor-1 (NRF-1) is the major transcription factor positively regulating the transcription of CXCR4. A number of signaling molecules have been shown to increase the transcription of CXCR4, such as elevated intracellular calcium, cyclic AMP, cytokines, like interleukin-2 (IL-2), IL-4, IL-7, IL-10, IL-15, TGF-1β, and growth factors such as basic fibroblast growth factor (bFGF), vascular endothelial growth factor (VEGF), and epidermal growth factor (EGF), and the role of these molecules in physiological responses after Cl₂ exposure remains unknown

Despite advances in treatment of lung injury resulting from exposure to Cl₂ and other toxic gases, there is still a scarcity of compounds and methods for treating Cl₂ damaged lungs that are safe and effective and that address the role of the SDF-1/CXCR4 chemokine/receptor axis in the migration, homing, and survival of leukocytes in the lung post exposure to gases including, but not limited to, Cl₂, Br₂, and phosgene. An ideal method would make use of a compound already approved by the FDA for other uses and would also be beneficial in mitigating lung injury after exposure to additional toxic gases. These needs and other needs are satisfied by the present disclosure.

### SUMMARY

In accordance with the purpose(s) of the present disclosure, as embodied and broadly described herein, the disclosure, in one aspect, relates to a method for treating or preventing at least one symptom of exposure to a toxic gas such as chlorine, bromine, or phosgene in a subject, the method including administering a composition containing AMD3100 or a pharmaceutically acceptable salt thereof to the subject. In one aspect, the composition can be administered by intramuscular injection, intranasally, or by inhalation in an amount of from about 0.01 to about 0.25 mg of AMD3100 per kg of subject body weight.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows a schematic of the process of lung inflammation starting with exposure to chlorine gas (Cl₂).

FIGS. 2A-2C show that SDF-1 levels are elevated in humans and animals exposed to Cl₂. Plasma SDF-1 levels were elevated in humans accidentally exposed to Cl₂ gas compared to matched non exposed humans (n=5) FIG. 2A: Male C57 BL/6 mice were exposed to air or Cl₂ (500 ppm, 30 min). Plasma (n=5-6) (FIG. 2B) and BALF (n=5-6) (FIG. 2C) SDF-1 was elevated in Cl₂ exposed mice 1 and 14 days post exposure. Values are mean±SEM. *P<0.05 vs. air by student's t test or by 1-way ANOVA with Tukey post-test.

FIGS. 3A-3B show CXCR4 expression is increased in lung and BALF leukocytes in animals exposed to Cl₂. Male C57 BL/6 mice were exposed to air or Cl₂ (500 ppm, 30 min). CXCR4 expression was elevated in whole lung tissue at 1 and 14 days post Cl₂ exposure (n=5-6, FIG. 3A). Fourteen days post Cl₂ exposure, CXCR4 expression was much higher in BALF leukocytes than blood leukocytes (n=4-5, FIG. 3B). Values are mean±SEM. *P<0.05 vs. air by student's t test or by 1-way ANOVA with Tukey post-test.

FIGS. 4A-4D show SDF-1/CXCR4 increases leukocyte migration and activity in the lungs of Cl₂ exposed mice. Adult male C57 BL/6 mice were exposed to air or Cl₂ (500 ppm, 30 min). One hour post exposure, air and some Cl₂ exposed mice received saline (IM), while other Cl₂ exposed animals received AMD3100 (IM). Mice were sacrificed 1 day post exposure. AMD3100 treated mice had lower BALF cell count compared to saline treated mice after Cl₂ exposure (n=5-6, FIG. 4A). AMD3100 reduced the migration of both macrophages and neutrophils into lungs (n=5-6, FIG. 4B). AMD3100 also reduced neutrophil elastase activity in BALF (n=5-6, FIG. 4C) and plasma (n=5-6, FIG. 4D) of mice exposed to Cl₂ gas. Values are mean±SEM. *P<0.05 vs. air+P 0.05 vs. Cl₂ saline by 1-way ANOVA with Tukey post-test.

FIGS. 5A-5H show AMD3100 attenuates Cl₂ induced acute lung injury. Adult male C57 BL/6 mice were exposed to air or Cl₂ (500 ppm, 30 min). One hour post exposure, air and some Cl₂ exposed mice received saline (IM), while other Cl₂ exposed animals received AMD3100 (IM). Mice were sacrificed 1 day post exposure. AMD3100 treated mice had lower BALF protein vs saline treated mice after Cl₂ exposure (n=5-6, FIG. 5A). AMD3100 reduced lung wet/dry weight ratio post Cl₂ exposure (n=5-6, FIG. 5B). Cl₂ exposure increased plasma levels of IL 6 (n=4, FIG. 5C) and TNF α (n=5, FIG. 5D) but not KC/GRO (n=3-4, FIG. 5E). AMD3100 reduced the plasma levels of these cytokines. AMD3100 also reduced Cl₂ induced increase in airway resistance (n=4-5, FIG. 5F), and lung injury score (n=5, FIG. 5G) upon H&E staining (FIG. 5H). Values are mean±SEM. *P<0.05 vs. air+P 0.05 vs. Cl₂ saline by 1-way ANOVA with Tukey post-test.

FIGS. 6A-6D show AMD3100 attenuates Cl₂ induced chronic lung injury. Adult male C57 BL/6 mice were exposed to air or Cl₂ (500 ppm, 30 min). One hour post exposure, air and some CO₂ exposed mice received saline (IM), while other Cl₂ exposed animals received AMD3100 (IM). Mice were sacrificed 14 days post exposure. AMD3100 treated mice had lower BALF protein vs saline treated mice after Cl₂ exposure (n=5, FIG. 6A). AMD3100 attenuated Cl₂ induced lung peribronchial fibrosis as evident by higher hydroxyproline content in lungs (n=4-5, FIG. 6B) and lung trichrome staining (black arrows, FIG. 6D). AMD3100 also attenuated Cl₂ induced alveolar septal damage as indicated by higher lung volumes on flexivent (n=4-5, FIG. 6C) and larger alveoli on H&E staining (FIG. 6D). Values are mean±SEM. *P<0.05 vs. air+P 0.05 vs. Cl₂ saline by 1-way ANOVA with Tukey post-test.

FIG. 7 shows a Kaplan-Meier curve indicating that AMD3100 improved survival in Cl₂ exposed mice Adult male C57 BL/6 mice were exposed to Cl₂ (500 ppm, 30 min). One hour post exposure, some Cl₂ exposed mice received saline (IM), while others received AMD3100 (IM). P<0.05 vs Cl₂+saline by log rank test.

FIGS. 8A-8E show heme increases CXCR4 expression in leukocytes. Adult humans exposed to Cl₂ gas had elevated plasma levels of cell free heme (n=5, FIG. 8A). Male C57 BL/6 mice exposed to Cl₂ (400 ppm, 30 min) had elevated plasma heme (n=9-10, FIG. 8B). Mice exposed to Cl₂ (500 ppm, 30 min) had elevated BALF levels of cell free heme on day 1 and 14 post exposure (n=5-6, FIG. 8C). Mice exposed to Cl₂ and then treated with either saline or hemopexin (Hx) 1 hour post, were sacrificed after 1 day. Hx reduced RBC fragility and hemolysis induced by Cl₂ (n=5-8, FIG. 8D). THP-1 cells challenged with hemin (25 μM, 24 h) had increased expression of CXCR4 (n=5, FIG. 8E). Values are mean±SEM. *P<0.05 vs. air or DMSO, P 0.05 vs. Cl₂ saline by student's t test or by 1-way ANOVA with Tukey post-test.

FIGS. 9A-9B show hypoxia increases CXCR4 expression in leukocytes. Adult SD rats exposed to Cl₂ (500 ppm, 30 min) had reduced oxygen saturation, which was restored by placing them in a chamber with 40% oxygen (FIG. 9A). THP-1 cells challenged with 3% oxygen for 24 hours had increased expression of CXCR4 (n=5, FIG. 9B). Values are mean±SEM. *P<0.05 vs. normoxia by student's t test.

FIGS. 10A-10C show Intranasal administration of AMD3100 attenuates Cl₂-induced acute lung injury. Adult male C57BL/6 mice were exposed to air or Cl₂ (500 ppm, 15 min). One hour post exposure, air and some Cl₂ exposed mice received saline (IN), while other Cl₂ exposed animals received AMD3100 (0.1 mg/kg body weight) (IN). Mice were sacrificed 1 day post exposure. AMD3100 treated mice had lower BALF protein versus saline treated mice after Cl₂ exposure (n=6-8) (FIG. 10A). AMD3100 treated mice had lower BALF inflammatory cell count compared to saline treated mice after Cl₂ exposure (n=6-8) (FIG. 10B). Lung wet/dry weight ratio did not increase significantly in AMD3100 treated animals compared to saline treated animals post Cl₂ exposure (n=6-7) (FIG. 10C). Values are mean±SEM. *P<0.05 by 1-way ANOVA with Tukey post-test.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

In one aspect, disclosed herein is method for treating or preventing at least one symptom of exposure to a toxic gas in a subject, the method including at least the step of administering a composition comprising AMD3100 or a pharmaceutically acceptable salt thereof to the subject. In one aspect, the pharmaceutically acceptable salt can be a hydrochloride salt. In another aspect, the toxic gas can be chlorine, bromine, phosgene, or any combination thereof.

In a further aspect, the at least one symptom can include difficulty breathing, cough, lung inflammation, hemolysis, increased plasma levels of cell-free heme, remodeling in large or small airways, obstruction in large or small airways, fibrosis, damage to alveolar septa, hypoxia, lung edema, protein extravasation into lungs, another decrease in lung function, or any combination thereof. In one aspect, the subject can be a mammal such as, for example, a human, rat, or mouse.

In an aspect, the composition can administered by intermuscular injection, intranasally, or by inhalation. In a further aspect, the composition can be administered in an amount comprising from about 0.01 to about 0.25 mg of AMD3100 per kg of subject body weight. In one aspect, the composition can be administered from 1 hour to 7 days following exposure to the toxic gas, or 1, 2, 3, 4, 5, 6, 7, 8, 9, 10, 11, or 12 hours, or 1, 2, 3, 4, 5, 6, or 7 days, or a combination of any of the foregoing values, or a range encompassing any of the foregoing values, where any number can be the upper or lower endpoint of the range. In any of these aspects, the composition can be administered one time.

In a further aspect, in the disclosed methods, at least one additional treatment can be administered to the subject. Further in this aspect, the at least one additional treatment can include administering an HIF-1 inhibitor, hemopexin, a VEGF inhibitor, a TGF-1βinhibitor, supplemental oxygen, or any combination thereof. In one aspect, the HIF-1 inhibitor can be topotecan, digoxin, digitoxin, temsirolimus, everolimus, bortezomib, or any combination thereof. In another aspect, the VEGF inhibitor can be sorafenib. In any of these aspects, the at least one additional treatment can be administered to the subject before, after, or simultaneously with the composition including AMD3100 or a pharmaceutically acceptable salt thereof.

In an aspect, performing the method can reduce a level of inflammatory cells, cell-free heme, CXCR4, SDF-1, or any combination thereof, in the subject in blood, plasma, bronchoalveolar lavage fluid, or any combination thereof, compared to an untreated subject exposed to the toxic gas. In another aspect, performing the method can reduce lung edema compared to an untreated subject exposed to the toxic gas.

Also disclosed herein are compositions for treating or preventing at least one symptom of exposure to a toxic gas in a subject, the composition including at least AMD3100 or a pharmaceutically acceptable salt thereof. In some aspects, the compositions can also include at least one additional therapeutic agent including, but not limited to, an HIF-1 inhibitor, hemopexin, a VEGF inhibitor, a TGF-1βinhibitor, or any combination thereof. Further in this aspect, the HIF-1 inhibitor can be topotecan, digoxin, digitoxin, temsirolimus, everolimus, bortezomib, or any combination thereof.

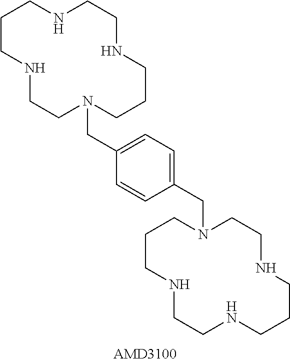 [SMILES: c1cc(CN2CCCNCCNCCCNCC2)ccc1CN1CCCNCCNCCCNCC1]

In one aspect, AMD3100 is also known as 1,1′-[1,4-phenylenebis-(methylene)]-bis-(1,4,8,11-tetraazacyclotetradecane). In some aspects, as used herein, AMD3100 can be a hydrochloride salt, wherein each molecule of AMD3100 is associated with up to 8 HCl molecules.

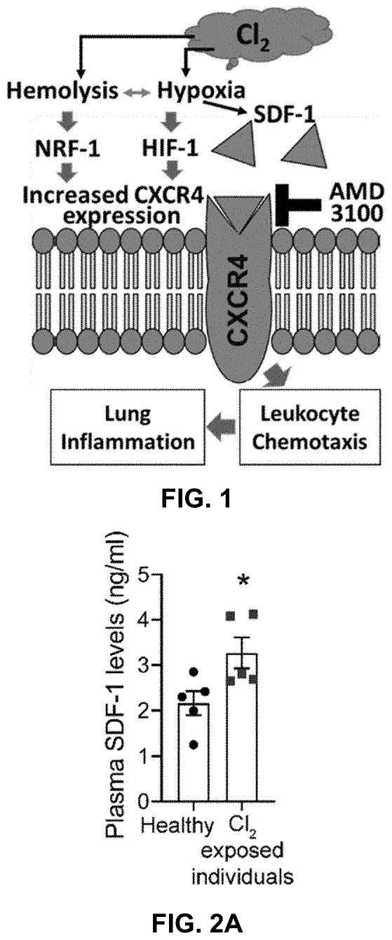

Both SDF-1 and CXCR4 levels are elevated in the lungs of Cl₂-exposed animals. Without wishing to be bound by theory, it is believed that the SDF-1/CXCR4 axis is involved in the migration, homing, and survival of leukocytes in lung after exposure to Cl₂ (FIG. 1). Further in this aspect, inhibiting this axis should attenuate Cl₂-induced lung morbidity and mortality.

In 2016, the American Association of Poison Control Centers reported over 6300 exposures to chlorine (Cl₂), making it the most common inhalational irritant in the United States 1.

However, current treatment strategies are insufficient and merely palliative. Acute and chronic lung injury post Cl₂ exposure is due to an uncontrolled activation of leukocytes—alveolar macrophages and sequestered neutrophils—in the lung. Excessive recruitment of leukocytes is critical to the pathogenesis of lung injury, and the magnitude and duration of the inflammatory process may ultimately determine the outcome in patients. In one aspect, AMD3100, an FDA-approved small molecule inhibitor of C-X-C chemokine receptor type 4 (CXCR4), which is an important leukocyte chemoattractant and is involved in the pathogenesis of acute and chronic injury due to various insults, is useful in the methods disclosed herein for treating or preventing at least one symptom of exposure to a toxic gas in a subject.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a leukocyte,” “a toxic gas,” or “a symptom of Cl₂ exposure,” includes, but is not limited to, mixtures or combinations of two or more such leukocytes, toxic gases, or symptoms of Cl₂ exposure, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In one aspect, in the disclosed methods, AMD3100 is administered intramuscularly to individuals who have been exposed to Cl₂ or another toxic gas.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be therapeutic in terms of a partial or complete cure of a disease, condition, symptom, or adverse effect attributed to the disease, disorder, or condition. The term “treatment” as used herein can include any treatment of exposure to Cl₂ and/or another toxic gas in a subject, particularly a human and can include any one or more of the following: (a) inhibiting the progression of lung damage post Cl₂ exposure, i.e., arresting its development; and (b) relieving one or more symptoms associated with lung damage post Cl₂ exposure.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

The term “pharmaceutically acceptable” describes a material that is not biologically or otherwise undesirable, i.e., without causing an unacceptable level of undesirable biological effects or interacting in a deleterious manner.

As used herein, “inflammatory cells” refers to cell types involved in an inflammatory response, for example, due to pathogen exposure, exposure to toxins, pollutants, irritants, or allergens, and/or physical or chemically-induced injury, including exposure to a toxic gas. Inflammatory cells in the lung include neutrophils, mast cells, eosinophils, macrophages, lymphocytes, and others. In one aspect, inflammatory cells may assist in the defense of damaged tissue against pathogens, may be involved in cellular repair processes, and/or another function.

Unless otherwise specified, pressures referred to herein are based on atmospheric pressure (i.e. one atmosphere).

### Pharmaceutical Compositions

In various aspects, the present disclosure relates to pharmaceutical compositions comprising a therapeutically effective amount of AMD3100 or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of AMD3100 or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for parenteral administration, although the most suitable route in any given case will depend on the particular host, and nature and severity of the conditions for which the active ingredient is being administered. In a further aspect, the disclosed pharmaceutical composition can be formulated to allow administration parenterally, intramuscularly, intravenously, or intradermally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In practice, AMD3100 or a pharmaceutically acceptable salt thereof can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, AMD3100 can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

AMD3100 is typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for intramuscular injection or parenteral administration. Carriers include solids or liquids, and the type of carrier is chosen based on the type of administration being used. The compounds may be administered as a dosage that has a known quantity of the compound.

It may optionally be necessary to stabilize a liquid (i.e., injectable) dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions, or suspensions, including saline and buffered media. Parenteral vehicles can include mannitol, normal serum albumin, sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the severity of the condition being treated (i.e., length of toxic gas exposure and concentration of toxic gas to which a subject was exposed); various factors specific to the medical history of the subject to whom the dosage is administered such as the age; weight, sex, extent of disorder and general physical condition of the particular subject, as well as other medication the individual may be taking; as is well known to those skilled in the art.

In one aspect, the pharmaceutical formulations including the disclosed compounds can be prepared as inhaled forms. In another aspect, the pharmaceutical formulations can be dispensed by metered-dose inhalers with or without adapter chambers, dry powder inhalers, nebulizers used with or without masks, sprays, or soft mist inhalers. In some aspects, when the pharmaceutical formulations are inhaled, they can include excipients especially useful for inhaled drugs including, but not limited to, sugars including lactose monohydrate or anhydrous lactose, lipids including oleic acid, amino acids, surfactants including lecithin, polymers, absorption enhancers, propellants, solvents, and the like. In some aspects, the pharmaceutical formulations can be micronized or spray dried or processed by another means prior to loading into dispensing devices. In another aspect, those skilled in the art will be able to select excipients to achieve the desired particle size, delivered dose, flowability, and other properties.

In one aspect, delivered dosages from aerosol or inhaled forms of the pharmaceutical compositions disclosed herein can be approximately the same as dosages for other forms (e.g., intramuscular injection). In an alternative aspect, delivered dosages from aerosol or inhaled forms of the pharmaceutical compositions disclosed herein can be higher than dosages for other forms, due to lack of mobility to other organs and systems. Without wishing to be bound by theory, delivery of the disclosed compounds directly to the lungs and airways by inhalation may reduce the dose of medication required to exert a protective effect on the airways due to direct contact of the disclosed compounds and/or any other active ingredients with the affected cells.

In another aspect, the aerosol and/or inhaled forms of the pharmaceutical compositions disclosed herein can be used once per day, or twice per day, or as needed as symptoms dictate. In a further aspect, the aerosol and/or inhaled forms of the pharmaceutical compositions disclosed herein can be used as single treatments or on a consistent basis until symptoms subside. In some aspects, aerosol and/or inhaled forms can be co-administered with parenteral therapies.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The disclosed pharmaceutical compositions can further comprise other therapeutically active compounds, which are usually applied in the treatment of the exposure to Cl₂, Br₂, phosgene, or another toxic gas.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered aspects, which should not be confused with the claims.

Aspect 1. A method for treating or preventing at least one symptom of exposure to a toxic gas in a subject, the method comprising administering a composition comprising AMD3100 or a pharmaceutically acceptable salt thereof to the subject.

Aspect 2. The method of aspect 1, wherein the toxic gas comprises chlorine, bromine, phosgene, or any combination thereof.

Aspect 3. The method of aspect 1 or 2, wherein the toxic gas is chlorine.

Aspect 4. The method of any one of aspects 1-3, wherein the at least one symptom comprises difficulty breathing, cough, lung inflammation, hemolysis, increased plasma levels of cell-free heme, remodeling in large or small airways, obstruction in large or small airways, fibrosis, damage to alveolar septa, hypoxia, lung edema, protein extravasation into lungs, another decrease in lung function, or any combination thereof.

Aspect 5. The method of any one of aspects 1-4, wherein the subject is a mammal.

Aspect 6. The method of aspect 5, wherein the mammal is a human, rat, or mouse.

Aspect 7. The method of any one of aspects 1-6, wherein the composition is administered by intermuscular injection.

Aspect 8. The method of any one of aspects 1-6, wherein the composition is administered intranasally or by inhalation.

Aspect 9. The method of any one of aspects 1-8, wherein the composition is administered from one hour to 7 days following exposure.

Aspect 10. The method of any one of aspects 1-9, wherein the composition is administered one time.

Aspect 11. The method of any one of aspects 1-10, wherein the composition is administered in an amount comprising from about 0.01 to about 0.25 mg of AMD3100 per kg of subject body weight.

Aspect 12. The method of any one of aspects 1-11, wherein the pharmaceutically acceptable salt of AMD3100 comprises a hydrochloride salt.

Aspect 13. The method of any one of aspects 1-12, further comprising administering at least one additional treatment to the subject.

Aspect 14. The method of aspect 13, wherein the at least one additional treatment comprises administering an HIF-1 inhibitor, hemopexin, a VEGF inhibitor, a TGF-1βinhibitor, supplemental oxygen, or any combination thereof.

Aspect 15. The method of aspect 14, wherein the HIF-1 inhibitor comprises topotecan, digoxin, digitoxin, temsirolimus, everolimus, bortezomib, or any combination thereof.

Aspect 16. The method of aspect 14, wherein the VEGF inhibitor comprises sorafenib.

Aspect 17. The method of any one of aspects 13-16, wherein the at least one additional treatment is administered to the subject simultaneously or sequentially with the composition comprising AMD3100 or the pharmaceutically acceptable salt thereof.

Aspect 18. The method of any one of aspects 1-17, wherein performing the method reduces a level of cell-free heme in the subject in plasma, bronchoalveolar lavage fluid, or both, compared to an untreated subject exposed to the toxic gas.

Aspect 19. The method of any one of aspects 1-18, wherein performing the method reduces a level of CXCR4 in the subject in blood, bronchoalveolar lavage fluid, or both, compared to an untreated subject exposed to the toxic gas.

Aspect 20. The method of any one of aspects 1-19, wherein performing the method reduces a level of SDF-1 in the subject in blood, bronchoalveolar lavage fluid, or both, compared to an untreated subject exposed to the toxic gas.

Aspect 21. The method of any one of aspects 1-20, wherein performing the method reduces a level of inflammatory cells in bronchoalveolar lavage fluid, compared to an untreated subject exposed to the toxic gas.

Aspect 22. The method of any one of aspects 1-21, wherein performing the method reduces lung edema compared to an untreated subject exposed to the toxic gas.

Aspect 23. A composition for treating or preventing at least one symptom of exposure to a toxic gas in a subject, the composition comprising AMD3100 or a pharmaceutically acceptable salt thereof to the subject.

Aspect 24. The composition of aspect 23, wherein the at least one symptom comprises difficulty breathing, cough, lung inflammation, hemolysis, increased plasma levels of cell-free heme, remodeling in large or small airways, obstruction in large or small airways, fibrosis, damage to alveolar septa, hypoxia, lung edema, protein extravasation into lungs, another decrease in lung function, or any combination thereof.

Aspect 25. The composition of aspect 23 or 24, wherein the composition reduces at least one of cell-free heme, CXCR4, or SDF-1 in the blood, plasma, bronchoalveolar lavage fluid, or any combination thereof.

Aspect 26. The composition of any one of aspects 23-25, wherein the pharmaceutically acceptable salt of AMD3100 comprises a hydrochloride salt.

Aspect 27. The composition of any one of aspects 23-26, further comprising at least one additional therapeutic agent.

Aspect 28. The composition of aspect 27, wherein the at least one additional therapeutic agent comprises an HIF-1 inhibitor, hemopexin, a VEGF inhibitor, a TGF-1p inhibitor, or any combination thereof.

Aspect 29. The method of aspect 28, wherein the HIF-1 inhibitor comprises topotecan, digoxin, digitoxin, temsirolimus, everolimus, bortezomib, or any combination thereof.

Aspect 30. The method of aspect 28, wherein the VEGF inhibitor comprises sorafenib.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Overview of Method to Determine Effect of AMD3100 on Lung Morbidity and Mortality Resulting from Cl₂ Gas Exposure

It has been found that SDF-1 is elevated in the plasma of humans accidentally exposed to Cl₂ gas. Mice exposed to Cl₂ also had high SDF-1 in plasma and bronchoalveolar lavage fluid (BALF), along with increased CXCR4 expression in alveolar leukocytes and peripheral lung tissue. The intramuscular administration of AMD3100 (0.05 mg/kg body weight) to mice, 1 hour post Cl₂ (500 ppm, 30 min) exposure, reduced the migration of leukocytes to lung and its associated acute inflammation (protein extravasation into lungs, lung edema, impaired respiratory mechanics). AMD3100 also prevented the chronic inflammatory sequelae in lungs (fibrotic and emphysematous changes) and improved survival in mice post C2 exposure. Therefore, it is believed that that SDF-1/CXCR4 axis is involved in the migration, homing, and survival of leukocytes in lung post exposure to C2 and therefore inhibiting this axis by AMD3100 would attenuate Cl₂-induced lung morbidity and mortality.

Establishing the role of SDF-1/CXCR4 axis in Cl₂-induced lung leukocyte migration, activation, and survival. Lung SDF-1/CXCR4 signaling cascade is elevated in mice post exposure to insults like lipopolysaccharide and bleomycin but its role in Cl₂ toxicity was previously unknown. First, it will be determined whether exposure to Cl₂ gas (500 ppm, 30 min) (a) increases SDF-1 concentration in BALF and the CXCR4 surface expression on the alveolar leukocytes (neutrophils and macrophages) and whole lung tissue over time and whether, (b) the SDF-1/CXCR4 axis is responsible for the migration of leukocytes from the vascular compartments to alveolar cavity, activation of leukocytes, and also survival of leukocytes in lung.

Optimizing the dosage regimen of AMD3100 for Cl₂ toxicity. The therapeutic regimen of AMD3100 as an anti-cancer drug in humans and as an anti-HIV-1 agent in animals is well known. However, the efficacy and potency of the clinically safe dosage (0.01-0.16 mg/kg) of AMD3100 in reducing acute and chronic lung injury and mortality in C57BL/6 mice, when administered intramuscularly one hour post Cl₂ (500 ppm, 30 min) exposure, must be determined. It must also be determined whether the systemic administration (intramuscular) vs. targeted delivery to lungs (intranasal) of AMD3100 has better therapeutic efficacy against Cl₂ toxicity.

Delineating the mechanisms of SDF-1/CXCR4 regulation post-Cl₂ exposure. The basal regulation of CXCR4 is mediated by the Nuclear Respiratory Factor-1 (NRF-1). In addition, the Hypoxia-Inducible Factor-1a (HIF-1a) augments CXCR4 expression in human leukocytes, endothelial, and cancer cells. HIF-1 has also been shown to upregulate the production of SDF-1 by endothelial cells. Hypoxia (low Pa_{O2}/Fi_{O2} ratio) was found in 58% of Cl₂ inhalation survivors of a 2005 South Carolina train derailment accident. In addition, it has previously been shown that the victims of Cl₂ exposure post-accident at the Birmingham water treatment plant in 2019 had hemolysis and elevated plasma levels of cell-free heme. Acute hemolysis can also exacerbate hypoxia and vice versa. Moreover, preliminary data in vitro showed that both heme and hypoxia upregulated CXCR4 in leukocytes. Therefore, it is hypothesized that post Cl₂, SDF-1 is upregulated by hypoxia-dependent HIF-1 activation, while CXCR4 expression is increased by cell-free heme mediated nuclear translocation of NRF-1 and also HIF-1 activation (FIG. 1).

### Example 2: Establishing the Role of the SDF-1/CXCR4 Axis in Cl₂-Induced Lung Leukocyte Migration, Activation, and Survival

Inflammatory response plays a critical role in the pathogenesis of lung injury post Cl₂ exposure. Cl₂-exposed mice develop lung inflammation characterized by infiltration of alveoli with neutrophils and prominent, large, foamy macrophages. After homing to the lung, neutrophils switch to an activated phenotype. The accumulation of neutrophils around the lung microvascular endothelial cells increases lung endothelial permeability, which was observed to be elevated in animals even 28 days post Cl₂ exposure. Mortality occurs primarily due to pulmonary edema with respiratory failure and circulatory collapse. SDF-1 is one of the prominent chemokines, which plays a crucial role in leukocyte accumulation at the site of lesion. However, the role of SDF-1 and its receptor, CXCR4, in the pathogenesis of Cl₂-induced lung injury is not known. Therefore, exploring the role of SDF-1/CXCR4 axis in leukocyte migration, homing, activation, and survival in lungs post exposure to C2 gas is an attractive strategy. The successful execution of this aim will support the hypothesis that blocking SDF-1/CXCR4 signaling is a critical therapeutic target to prevent Cl₂ toxicity.

In a recent study, in a mouse model of LPS-induced lung injury, the surface expression of CXCR4 was elevated in neutrophils. Further, in a rat model of acute lung injury post cardiopulmonary bypass, SDF-1 was increased in blood and BALF, and CXCR4 expression was high in lung tissue and isolated neutrophils from blood. Similarly, an intratracheal instillation of bleomycin in C57BL/6 mice increased SDF-1 in serum and bronchial alveolar lavage fluid (BALF). These changes were accompanied by increased numbers of CXCR4+ cells in the lungs. The study also found that the lung tissue from patients with idiopathic pulmonary fibrosis had higher numbers of cells expressing both SDF-1 and CXCR4 than did normal lungs. Therefore, as a first step in establishing the role of SDF-1/CXCR4 in Cl₂-associated lung inflammation, the SDF-1 concentrations in BALF and plasma and CXCR4 expression in lung cells, whole lung tissue, peripheral immune cells isolated from BALF, and blood, were measured over time in mice post exposure to Cl₂ gas. It is postulated that lung leukocytes would have much higher expression of CXCR4 then blood leukocytes post Cl₂ gas exposure, which would increase the migration of circulating leukocytes towards Cl₂ exposed lungs.

Previous work has demonstrated that Cl₂ exposure increases the migration, homing, and activation of leukocytes (neutrophils and macrophages) into lungs. Although leukocyte activation is vital for the host defense, overzealous activation leads to tissue damage by release of cytotoxic and immune cell-activating agents such as proteinases, cationic polypeptides, cytokines, and reactive oxygen species. Importantly, the concentration of leukocytes in BALF correlates with severity of acute respiratory distress syndrome (ARDS) and outcome in patients, whereas the severity of lung injury has been shown to be reduced by leukocyte depletion in mice. It still needs to be determined whether leukocytes with high SDF-1/CXCR4 post C2 exposure are more active and have increased capability to migrate and survive, which would justify the use of CXCR4 antagonist as a therapy against Cl₂-induced inflammation. It is postulated that high SDF-1/CXCR4 would enhance leukocyte ability to produce reactive species and survive post C2 exposure.

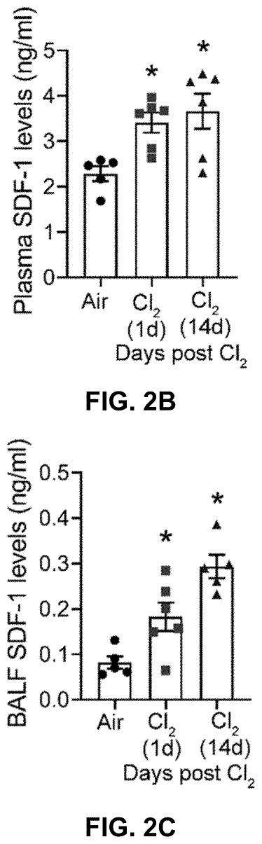

SDF-1 levels are elevated in humans and mice post C2 gas exposure. Plasma SDF-1 was measured in 5 adult humans, 2 days after they were admitted to the University of Alabama at Birmingham Emergency Department, post accidental exposure to Cl₂ gas at the Birmingham water treatment plant in 2019. The average age of exposed humans was 48 years with 80% of them being males. The study was approved by the University of Alabama at Birmingham Institutional Review Board (IRB Protocol 300002065 and 300000860). Blood was also collected from corresponding age and sex matched non exposed humans. Plasma SDF-1 levels were significantly increased from 2000 pg/mL to 3000 pg/mL in Cl₂ gas exposed patients compared to their sex- and age-matched non-exposed individuals (FIG. 2A). These levels of SDF-1 are comparable to what were found in the plasma of patients with community acquired pneumonia. Similarly, plasma (FIG. 2B) and BALF (FIG. 2C) SDF-1 levels were elevated in adult male C57BL/6 mice, 1 day post exposure to Cl₂ gas (500 ppm, 30 min). Interestingly, the SDF-1 levels in mice remained elevated even 14 days post C2 exposure.

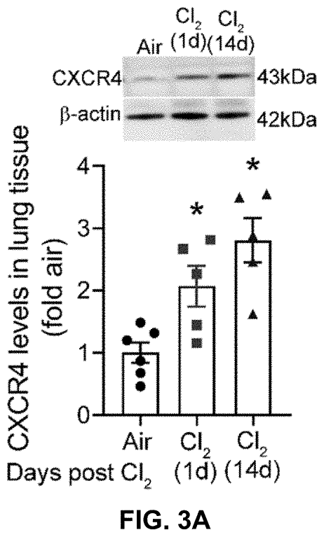

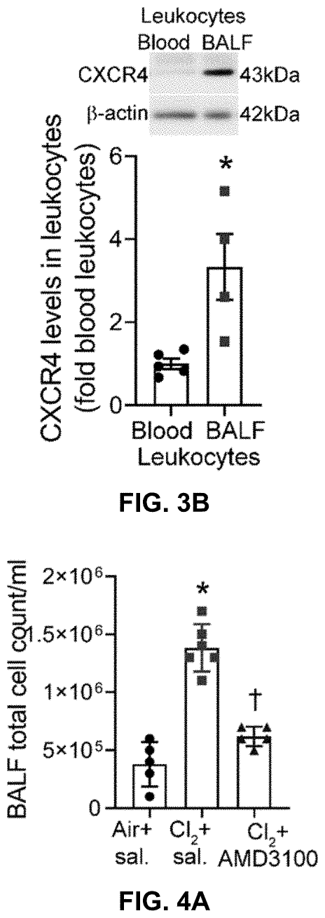

CXCR4 expression is increased in the whole lung tissue and BALF leukocytes in Cl₂ exposed mice. Adult male C57BL/6 mice were exposed to air or Cl₂ (500 ppm, 30 min) and then lungs were harvested on day 1 or 14 post exposure. Immunoblotting showed increased expression of CXCR4 in lungs on day 1 and 14 post exposure (FIG. 3A). Immunoblotting also showed that the expression of CXCR4 was significantly higher in the leukocytes recovered from BALF than the leukocytes isolated from blood 14 days post Cl₂ (500 ppm, 30 min) exposure (FIG. 3B). Taken together, these findings suggested that Cl₂ exposure increases CXCR4 expression in lung leukocytes more than blood leukocytes. This higher lung CXCR4 gradient along with SDF-1 would enhance leukocyte chemotaxis towards lung and promote lung inflammation.

Animals. Adult C57BL/6 mice (25-30 gram body weight, both sexes in equal number) were used for the study. All mice were housed in conventional polycarbonate cages with woodchip bedding under a 12 hour/12 hour light/dark cycle with ad libitum access to a standard diet and water. Euthanasia protocol based on intraperitoneal injections of ketamine and xylazine was used in the study for mice to minimize pain and distress. All animal care and experimental procedures were approved by the Institutional Animal Care and Use Committee at the University of Alabama in Birmingham.

Cl₂ exposure. Mice were exposed to air or Cl₂ (500 ppm) for 30 minutes in environmental chambers and then returned to room air. Body weight and peripheral O₂ saturation were measured with a mouse Paw Pulse Oximeter Sensor over time. Mice were sacrificed at different intervals (1, 7, 14 day) post air or Cl₂ exposure to determine the protein levels of SDF-1 (in BALF, plasma, lung cells and whole lung tissue) and CXCR4 (in leukocytes from BALF and blood, and also from isolated lung cells and whole lung tissue).

Isolating single cell suspension from whole lung. Mouse lungs were perfused with PBS via the right ventricles. PBS-perfused lungs were isolated with other mediastinal organs. Dispase II solution were instilled into the lungs through the trachea, which was ligated with a silk suture. After incubation at 37° C. for 50 min, the lungs were separated from the other mediastinal organs. The lungs were thoroughly minced and digested in PBS with 0.1% collagenase, 0.01% deoxyribonuclease I, and 5 mM CaCl₂ at 37° C. for 20 min. The cells were suspended in red blood cell lysing buffer to remove red blood cells and subsequently washed with PBS. The cells were then centrifuged and resuspended in PBS for SDF-1 and CXCR4 quantification.

Isolation of BALF and blood leukocytes. Plasma and BALF were isolated from mice according to a published procedure. To isolate blood leukocytes, blood drawn from mice was mixed with an equal volume of 3% dextran for 30 min to separate leukocyte-rich plasma. Leukocytes containing supernatant were centrifuged (10 min, 1000 rpm, 40° C.). The supernatant was discarded, and the remaining RBC and leukocytes were collected. The RBCs were lysed by adding hypotonic solution and leukocytes will be re-suspended in 1×PBS/glucose. Similarly, BALF leukocytes from mouse were isolated according to a published procedure.

SDF-1 protein quantification. Mouse SDF-1 levels were quantified using the Quantikine mouse CXCL12/SDF-1a ELISA kit. Results were reported as picograms of SDF-1 per milligram of total protein. Plasma, BALF, lung cells and whole lung tissue lysate were collected as detailed above and analyzed for SDF-1 protein.

CXCR4 protein quantitation. Cells and tissue collected above were mixed with PBS with 1×protease inhibitor mixture and homogenized on ice. Homogenates were sonicated and then centrifuged for 10 min at 2000×g. The resulting supernatants were carefully aspirated and the total protein concentrations were determined by Bradford assay. Supernatants were then assayed for CXCR4 protein levels by immunoblotting.

Evaluation of surface expression of CXCR4 in cells. The surface CXCR4 expression in leukocytes isolated from lung and blood, and lung single cell suspension was evaluated by staining the cells with a FITC-labeled anti-CXCR4 antibody and then analyzing using a FACSCalibur flow cytometer.

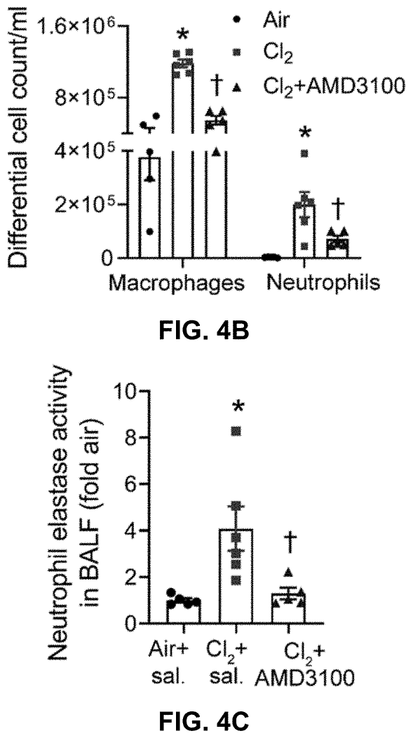

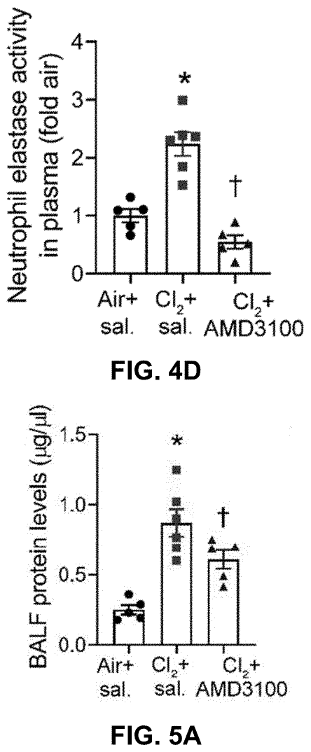

SDF-1/CXCR4 signaling increases leukocyte migration and activity in lung post Cl₂ exposure. To determine the role of SDF-1/CXCR4, adults male C57BL/6 mice were exposed to air or Cl₂ gas (500 ppm, 30 min) and then were administered the CXCR4 antagonist, AMD3100 (0.05 mg/kg body weight), intramuscularly 1 hour post exposure. The analysis of cells in the alveolar cavity 1 day post exposure demonstrated increased accumulation of cells in BALF of Cl₂ exposed mice (FIG. 4A). Further, the differential counting of the cells demonstrated increased migration of both macrophages and neutrophils into lungs post Cl₂ exposure (FIG. 4B). In addition, higher neutrophil elastase activity was seen in BALF (FIG. 4C) and plasma (FIG. 4D) of Cl₂ exposed mice. The animals that received an intramuscular injection of AMD3100 had reduced BALF total cell count, macrophages, and neutrophils, and also neutrophil elastase activity. Together, these results indicated that SDF-1/CXCR4 signaling is responsible for leukocyte migration and activity post Cl₂ exposure. It is also proposed to perform an in vitro neutrophil migration, activation, and survival assay as mentioned below.

Further Animal Studies. C57BL/6 mice were exposed to air or Cl₂ as described previously. Neutrophils were separated from BALF cells and single cell suspension from whole lung tissue as above using an anti-Ly6G (1A8) MicroBead Kit according to the manufacturer's protocol. The purity of the isolated neutrophils was evaluated using both microscopic and flow cytometric analyses

Neutrophil migration assay. Neutrophils were placed on a modified Boyden chamber with 3-μm pores to evaluate the migration upon stimulation by SDF-1 (different concentrations). Splenocytes, which can migrate toward SDF-1, were used as a positive control. A total of 5×10⁵ neutrophils or splenocytes in 200 μL of RPMI 1640 medium containing 0.25% bovine serum albumin were placed in the upper chambers. The upper chambers were then placed in individual wells of a 24-well cell culture plate containing 500 μL of assay buffer either with or without mouse SDF-1 (50 nM). An equal number of neutrophils or splenocytes were added to some of the lower wells without a top chamber to provide a standard count of total cells. In some experiments, the cells were pre-incubated with 100-nM AMD3100 at 37° C. for 30 min. The chambers were incubated for 2 h at 37° C. The cells in the lower chamber were collected and the % of migration determined from the original cell input.

Neutrophil activation. Lung neutrophils were preincubated with either the CXCR4 antagonist, AMD3100 (100 nM), or an appropriate vehicle for 1 h at 37° C. The neutrophils were then mixed with 100 ng/mL SDF-1 and incubated for an additional 4 h at 37° C. The activity of isolated neutrophils was evaluated by measuring (a) NADPH oxidase derived reactive species, (b) myeloperoxidase activity, (c) neutrophil extracellular traps (NETs) formation, and (d) neutrophil elastase activity using commercially available assay kits as per manufacturer's recommendations.

Cell death analysis. Lung neutrophils (2×10⁶) in RPMI 1640 containing 10% serum were either untreated or preincubated with the CXCR4 antagonist (AMD3100 100 nM) for 1 h at 37° C. The neutrophils were then mixed with 100 ng/mL SDF-1 and incubated for an additional 24 h at 37° C. After this incubation, the neutrophils were counted and the percentage of dead cells calculated using trypan blue staining. Neutrophils were also stained with Annexin V and 7-aminoactinomycin D (7-AAD), and then analyzed using a flow cytometer for viability.

Preliminary Results. In preliminary data, (FIG. 2C), there was a mean difference of 0.101 in BALF SDF-1 levels between mice exposed to air vs. Cl₂ (1 day post) with a pooled standard deviation of 0.061. Assuming a 5% significance level and a sample size of 20 (10 air, 10 Cl₂), a two-sample t-test has 95% power to detect a significant difference in BALF SDF-1 levels in these 2 groups. Power for future experiments was calculated based on descriptive statistics. Statistical significance was determined by either two-sample t-test (for 2 groups) or one-way ANOVA (for 3 or more groups) with Tukey's post-test. Depending on whether groups have equal variances, data may need to be transformed logarithmically. Total mice needed=240.

C57BL/6 mice exposed to Cl₂ gas exhibited increased levels of SDF-1 in the plasma and BALF. SDF-1 levels were higher in the alveolar leukocytes compared to the blood leukocytes. The expression of CXCR4 was also higher in the lung cells and alveolar leukocytes than the blood leukocytes. This led to increased migration, activation, and survival of leukocytes in response to SDF-1 stimulation. SDF-1/CXCR4 levels to remain elevated even 14 days post Cl₂ exposure and promote the development of chronic fibrotic and emphysematous changes in lungs due to increased production of reactive species and elastases by neutrophils. This is consistent with previous findings that lung SDF-1/CXCR4 were seen to be elevated in mice post exposure to insults like lipopolysaccharide and bleomycin and also in lung tissue from patients with idiopathic pulmonary fibrosis. Interpretation.

Though, it is widely believed that SDF-1 and CXCR4 are a relatively monogamous ligand-receptor pair, few studies have suggested that extracellular ubiquitin can also bind CXCR4 and induce chemotaxis in vitro, although it appears less potent than SDF-1. Ubiquitin and CXCR4 binding was also blocked by AMD3100. The binding of CXCR4 to ubiquitin can target CXCR4 for lysosomal degradation. The macrophage migration inhibitory factor (MIF) has also been shown to interact with CXCR4 and is considered a partial allosteric agonist of CXCR4. MIF dependent activation of CXCR4 can also be inhibited by AMD3100. In addition, the chemokine receptor, CXCR7, has been shown to bind SDF-1. However, the increased expression of CXCR7 post lung injury was only transitory, unlike CXCR4, which is elevated in lungs for at least 14 days (FIGS. 3A-3B) post Cl₂ exposure. In addition, the role of SDF-1/CXCR7 signaling is controversial, with some studies showing that this signaling promotes tissue repair in acute lung injury and lung fibrosis, while others have shown that they promote lung injury. Therefore, if increased expression of CXCR7 is found in the lung after Cl₂ exposure, an alternative approach would be to use CXCR7 antagonist, CCX771. However, based upon data acquired thus far, it is believed that that SDF-1/CXCR4 signaling cascade in the predominant chemoattractant for leukocytes post Cl₂ exposure.

### Example 3: Optimizing the Dosage Regimen of AMD3100 for Cl₂ Toxicity

AMD3100 is a well-documented specific antagonist of CXCR4 and widely applied as SDF-1/CXCR4 blocker. The therapeutic utility of AMD3100 as an anti-cancer drug in humans is well documented, where AMD3100 has been clinically useful in stopping the directional trafficking and invasion of cancer cells to sites of metastases. In HIV, AMD3100, has been shown to inhibit the entry of HIV-1 virus into cells via the CXCR4 co-receptor. Similarly, AMD3100 attenuated the allergic lung inflammation and airway hyperreactivity in a mouse model of asthma by reducing peribronchial eosinophilia. Further, in a mouse model of bleomycin-induced lung fibrosis, AMD3100 reduced lung collagen content, inhibited the migration of fibrocytes in response to SDF-1 in vitro, and reduced the trafficking of fibrocytes into the lungs treated with bleocmycin in vivo. In neonatal rats, AMD3100 reduced hyperoxia-induced lung injury by improving alveolarization, and decreasing BALF macrophage and neutrophil count and reducing lung myeloperoxidase activity. Finally, in a mouse model of LPS-induced lung injury, the administration of AMD3100 decreased transendothelial and transepithelial migration of immune cells. For these reasons, it was hypothesized that AMD3100 may have a therapeutic potential in reversing inhalation lung injury caused by toxic gases such as Cl₂, where the migration of neutrophils and macrophages in lung plays a critical role in lung inflammation and mortality. However, the efficacy and potency of the clinically safe dosage (0.01-0.16 mg/kg) of AMD3100 in reducing Cl₂ toxicity in mice, when administered intramuscularly one hour post Cl₂ (500 ppm, 30 min) exposure, have not been determined.

Current treatment for Cl₂ gas toxicity includes supplemental oxygen, bronchodilators, and antibiotics in case of infection. Assisted or supported ventilation with tracheal intubation and positive pressure ventilation may also be necessary, increasing the risk of lung injury and bacterial lung infections. Therefore, there is an urgent need to develop new therapeutic agents, which are based on etio-pathological findings in patients and animals exposed to C2 gas. As shown in FIGS. 2A-3B, humans and animals exposed to C2 have elevated SDF-1 and increased lung expression of CXCR4, which is a known chemoattractant for leukocytes. Therefore, the significance of this aim is that the use of AMD3100 to reverse Cl₂-induced acute lung injury is based upon the etio-pathological changes observed after Cl₂ exposure. The efficacy of AMD3100, when administered systemically (intramuscular) vs local administration into the lungs (intranasal) should also be determined. It is postulated that both intramuscular and intranasal administration of AMD3100 will mitigate Cl₂-induced acute lung inflammation in mice.

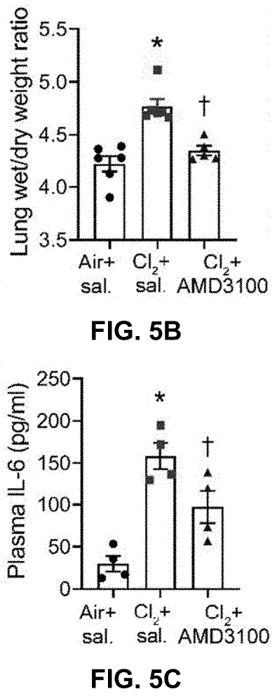

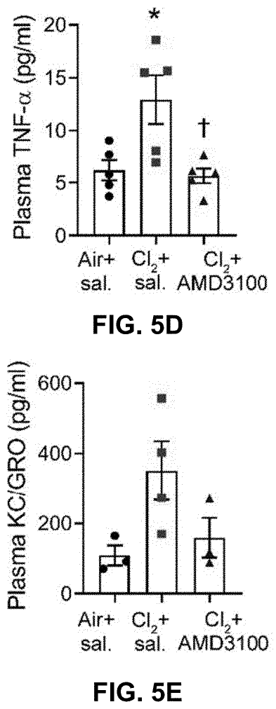

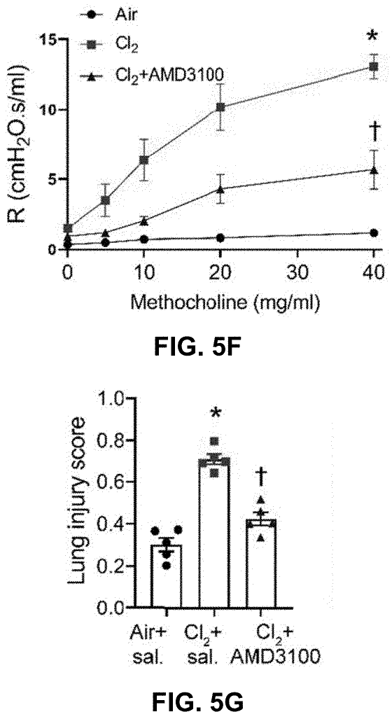

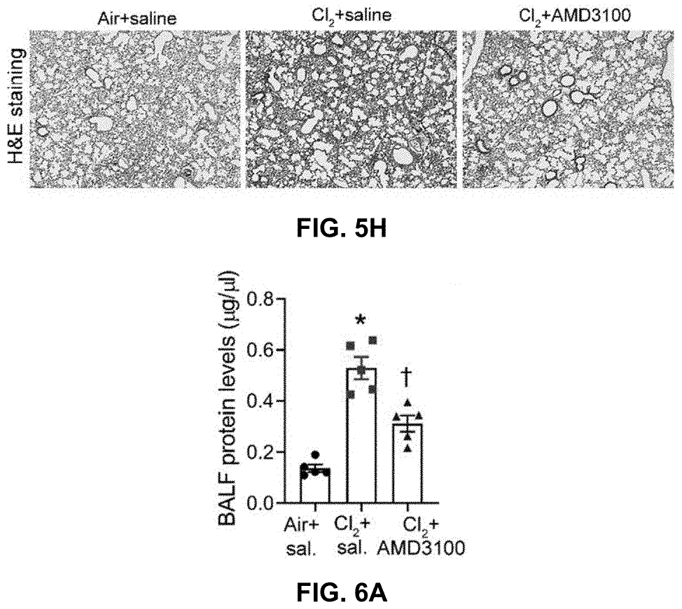

Intramuscular injection of AMD3100 attenuates Cl₂-induced acute lung injury. Adult male C57BL/6 mice were exposed to air or Cl₂ gas (500 ppm, 30 min). Several Cl₂ exposed animals received an intramuscular injection of saline or the CXCR4 antagonist, AMD3100 (0.05 mg/kg body weight), 1 hour post exposure. Air exposed mice received saline. One day later, the analysis of BALF demonstrated elevated protein content (sign of endothelial barrier dysfunction) in Cl₂ exposed animals that received saline but not in animals that were given AMD3100 (FIG. 5A). In another subset of animals, the lung wet/dry weight ratio was elevated (sign of lung edema) post Cl₂ exposure in saline treated animals but not in AMD3100 treated mice (FIG. 5B). AMD3100 also reduced cytokines IL-6 (FIG. 5C) and TNF-α (FIG. 5D) in plasma of Cl₂ exposed animals. However, plasma KC/GRO (FIG. 5E) was not significantly higher after Cl₂ exposure. Further, the measurement of lung mechanics by FlexiVent showed that Cl₂ exposure increased total lung resistance following challenge with methacholine (FIG. 5F). However, AMD3100 treated mice had significantly lower lung resistance compared to saline treated animals. The histological examination demonstrated that AMD3100 mitigated Cl₂-induced increase in lung injury score (FIG. 5G) in lung sections stained with hematoxylin & eosin (FIG. 5H). Taken together, these results indicate that CXCR4 is an important target for therapeutics against Cl₂-induced acute lung injury.

Animals. Adult C57BL/6 mice (25-30 g, both sexes) were used for the study.

Cl₂ exposure. Mice were exposed to air or Cl₂ (500 ppm) for 30 minutes in environmental chambers and then returned to room air. Body weight and peripheral O₂ saturation were measured with a Mouse Paw Pulse Oximeter Sensor over time.

AMD3100 administration. Mice received a single intramuscular injection of clinically safe dosage (0.01-0.16 mg/kg) of AMD3100 or saline, 1 hour post exposure to Cl₂. (This dose of AMD3100 did not cause any side effects in humans even after daily administration for 6 months). Up to 3 different doses (0.01, 0.05, and 0.1 mg/kg) of AMD3100 were tested initially to measure BALF protein and cell count and ultimately the most suitable dose was picked to perform other parameters of acute lung injury. Several mice will be given intranasal AMD3100 (in 50 μL saline) to determine the effectiveness of intranasal administration of AMD3100 in limiting Cl₂-induced acute lung injury.

Assessment of acute lung injury was done according to a previously published procedure. Mice exposed to Cl₂ (500 ppm, 30 min) were sacrificed 1 day post exposure, as these rodents developed significant lung morbidity within 1 day. Lungs were lavaged and plasma protein content and cell count (total and differential) were determined in BALF. Cytokines and chemokines will be quantified in BALF and plasma according to a previously published procedure. Lungs were micro-dissected, paraffin processed, stained with H&E and myeloperoxidase, imaged, and lung injury scored, according to the guidelines laid down in the American Thoracic Society workshop report. Lung edema was assessed by the lung wet to dry weight ratio. Blood gases were measured in the abdominal aorta blood by the OPTI® CCA-TS Blood Gas and Electrolyte Analyzer (OPTIMedical) with E-BUN cassettes. Respiratory mechanics were assessed by FlexiVent.

Some individuals exposed to Cl₂ experience a full recovery from acute injury, whereas others develop persistent adverse effects, such as respiratory symptoms, inflammation, and lung-function decrements. In animal models, Cl₂ can produce persistent inflammation, remodeling, and obstruction in large or small airways, depending on species. Distal airways, which lack basal cells are repaired less efficiently, and are prone to chronic inflammation, which may lead to fibrosis and damage to alveolar septa. The chronic sequelae of Cl₂ exposure may be even more pronounced among those survivors that are older, have smoked, and/or have pre-existing chronic lung disease. Persistent Cl₂-induced airway disease in humans is treated with asthma medication to relieve symptoms. However, such treatment does not ameliorate the underlying disease pathogenesis, so treatments that are more effective at preventing initial development of airway disease after irritant gas exposure are needed. It is postulated that AMD3100 will attenuate Cl₂-induced chronic lung inflammation in mice.

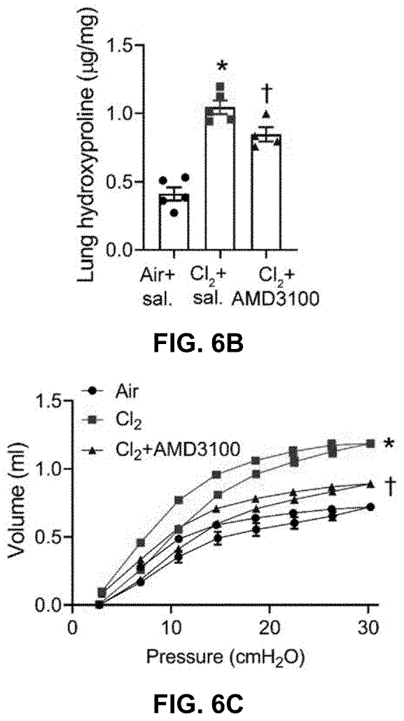

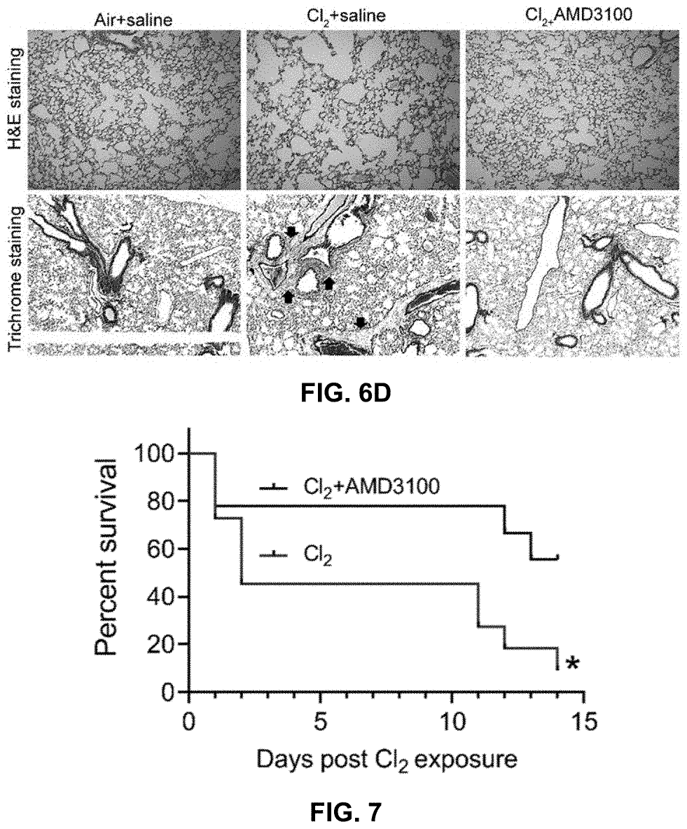

Intramuscular administration of AMD3100 attenuates Cl₂-induced chronic lung injury and mortality. BALF and plasma SDF-1 levels (FIGS. 2B-2C) and the expression of CXCR4 in whole lung tissue and BALF leukocytes (FIGS. 3A-3B) were elevated even 14 days post Cl₂ exposure. Therefore, to investigate whether these SDF-1/CXCR4 levels were responsible for the development of chronic lung inflammation and associated respiratory dysfunction, adult male C57BL/6 mice (25-30 g) were exposed to air or Cl₂ gas (500 ppm, 30 min) and returned them to room air. Several Cl₂ exposed animals received an intramuscular injection of saline or AMD3100 (0.05 mg/kg body weight), 1 hour post exposure. Air exposed mice received saline. Fourteen days later, the protein content was still elevated in the BALF of Cl₂ exposed animals that received saline but not in animals that were given AMD3100 post Cl₂ exposure (FIG. 6A) suggesting that AMD3100 attenuates chronic lung inflammation. To determine if chronic inflammation correlated with fibrotic and emphysematous changes in lung phenotype, lung markers of fibrosis and alveolar damage were measured. Cl₂ exposed mice treated with saline had increased lung parenchymal hydroxyproline levels on day 14 post exposure (FIG. 6B) suggesting increased collagen levels in the lungs. These fibrotic changes were accompanied by histological observation, where Mason's trichrome staining of the lung demonstrated significant accumulation of collagen in the lungs, primarily around bronchioles on day 14 post exposure (FIG. 6D). Upon staining the lungs with hematoxylin and eosin (H&E) (FIG. 6D), it was found that on day 14 post Cl₂ gas inhalation, there was alveolar wall destruction and airspace enlargement. These changes were corroborated with the measurements of pressure-volume (PV) relationships in anesthetized mice with flexiVent, which demonstrated that Cl₂ exposure resulted in increased lung volumes (FIG. 6C) as an indicator of emphysematous phenotype. The role of leukocytes in these chronic lung changes is highlighted by data showing that neutrophil elastase activity was elevated in BALF and plasma of Cl₂ exposed mice even 14 days post exposure (FIGS. 4C-4D). The release of proteinases such as elastase by macrophages and neutrophils plays a significant role in alveolar wall destruction. The inhibition of neutrophil elastase activity in mice that received AMD3100 post exposure to Cl₂, decreased lung hydroxyproline, attenuated peribronchial collagen deposition, reduced lung volumes, and mitigated lung alveolar destruction. Finally, 1 hour post Cl₂ exposure, an intramuscular injection of AMD3100 reduced overall mortality in C57BL/6 mice (FIG. 7).

Further Animal Studies. Adult C57BL/6 mice (25-30 g, both sexes) were used for the study. Mice were exposed to air or Cl₂ (500 ppm) for 30 minutes.

AMD3100 administration. Mice received a single intramuscular injection of AMD3100 or saline, 1 hour post exposure to Cl₂. It was determined whether the late administration of AMD3100 (1 or 7 days post Cl₂ exposure) prevented the development of chronic lung morbidity and mortality in mice. Several mice will be given AMD3100 intranasally (in 50 μL saline) to determine the effectiveness of targeted (lung) administration of AMD3100 in limiting Cl₂-induced chronic lung injury.

Assessment of chronic lung injury was conducted according to a published procedure. Mice exposed to Cl₂ (500 ppm, 30 min) were sacrificed 14 days post exposure. Lungs were stained with Masson's trichrome to assess collagen and with anti-α-smooth muscle actin antibody to determine myofibroblast content. Lung collagen levels were measured by the hydroxyproline levels. Alveolar septal damage was determined by staining with H&E and measuring the mean linear intercept (Lm) to assess the size of air space. Increased Lm indicated emphysema-like phenotype. Lm was measured by dividing the total length of line drawn across 20 randomly selected lung fields by the number of intercepts (alveolar septum) at 200× magnification. Alveoli number was determined by the number of measurements made for the Lm. The activity of neutrophil elastases, which can damage alveolar septa, was measured using ELISA in the BALF and plasma. The assessment of lung pressure-volume curve was assessed by FlexiVent. A pressure-volume loop generated vital (total) lung capacity (A), inspiratory capacity from zero pressure (B), form of deflating PV loop (K), static compliance (C), static elastance (E), and hysteresis.

Survival studies. Male and female C57BL/6 mice exposed to Cl₂ (500 ppm, 30 min) with or without AMD3100 administration were monitored for 30 days. Animals were sacrificed if body weight dropped by 30% according to IACUC guidelines. Survival was analyzed by the Kaplan-Meier method. Statistical significance was analyzed by the log-rank test.

In preliminary experiments, (FIG. 5A), there was a mean difference of 0.2627 in BALF protein levels between mice exposed to Cl₂+saline vs. Cl₂+AMD3100 with a pooled standard deviation of 0.202409. Assuming a 5% significance level and a sample size of 20 (10 Cl₂, 10 Cl₂+AMD3100), a two-sample t-test had 80% power to detect a significant difference in BALF protein levels between these 2 groups. Power for future experiments was calculated based on descriptive statistics. Statistical significance was determined by either two-sample t-test (for 2 groups) or one-way ANOVA (for 3 or more groups) with Tukey's post-test. Data was transformed logarithmically in some instances. Total mice needed=520.

Based upon preliminary findings in Cl₂ exposed mice and published data in Cl₂, bromine, and phosgene exposed mice, it was anticipated that the BALF protein and neutrophils, Paco₂, central and peripheral airway resistance, and lung wet to dry weight ratio would be elevated in mice exposed to Cl₂ 1 day post exposure. In chronic lung injury studies, it was anticipated that mice exposed to Cl₂ would have increased collagen deposition, higher plasma and lung elastase levels and activity, and damage to alveolar septa 14 days post exposure. It was further anticipated that administration of AMD3100 intramuscularly and intranasally would improve lung injury and mortality in these animals.

Interpretation. The anticipated results suggest that SDF-1/CXCR4 axis plays a critical role in Cl₂-induced acute and chronic lung inflammation. Importantly, if the late administration of AMD3100 (7 days post Cl₂) is able to reverse the pathological development of chronic lung phenotype, CXCR4 antagonism may be an important therapeutic strategy to prevent late manifestations in lungs of patients who have already developed acute lung injury.

Alternative outcomes. Since several chemokines, including CXCL-8, CXCL-1, CXCL-5, and CCL-2, are elevated in the BALF of patients with acute lung injury; their levels may correlate with outcome. In animal models of acute lung injury, the levels of these chemokine are elevated in the lungs, and neutralization of chemokines using antibodies or their receptors using knock out mice have shown to attenuate injury. Therefore, it is entirely possible that these chemokines are also elevated in the lungs of Cl₂ exposed mice. For instance, the keratinocyte-derived chemokine KC/CXCL1 has been shown to be elevated in lungs of mice post Cl₂ exposure. In preliminary data, also found elevated levels of KC were also found (although not significant due to low ‘n’ value) in Cl₂ exposed mice (FIG. 5D). KC is a strong chemoattractant for neutrophils and therefore its role in lung inflammation post Cl₂ cannot be discounted. Mice that received AMD3100 had lower KC levels, probably due to reduced number of inflammatory cells.

### Example 4: Mechanisms of SDF-1/CXCR4 Regulation Post Cl₂ Exposure

Further study of the mechanisms of SDF-1/CXCR4 regulation upon exposure to Cl₂ gas was performed in order to identify alternative therapeutic targets that may mitigate Cl₂ toxicity. For instance, NRF-1 and HIF-1 are known to upregulate the expression of CXCR4 and therefore, if in HIF-1 is found to play a significant role in CXCR4 mediated leukocyte chemotaxis post Cl₂ exposure, some of the known FDA-approved HIF-1 inhibitors including topotecan, cardiac glycosides, temsirolimus, everolimus, and bortezomib may serve as alternative therapeutics in the future. Similarly, preventing hypoxia in patients by giving supplemental oxygen may limit HIF-1 dependent upregulation of CXCR4 or in combination with AMD3100 may have a synergistic response in limiting Cl₂ toxicity. Likewise, it has previously been demonstrated that exposure to toxic gases such as Cl₂, bromine, or phosgene cause hemolysis and increased plasma and BALF levels of cell-free heme in mice and humans. The scavenging of cell-free heme in mice post toxic gas inhalation reduced plasma and BALF levels of cell-free heme and also attenuated the migration of leukocytes into the injured lung. It is therefore hypothesized that post Cl₂ exposure, heme-mediated upregulation of NRF-1 increases CXCR4 expression in the lung, leading to leukocyte chemotaxis, activation, and survival. If this hypothesis is shown to be correct, it would identify cell-free heme as an alternative therapeutic target to lower SDF-1/CXCR4 cascade in combination with AMD3100, HIF-1 inhibitors, and oxygen supplementation.

Role of heme in regulating SDF-1/CXCR4 signaling post Cl₂ exposure. Cell-free heme is an important injurious agent in several pathologies such as the endothelial injury after lipopolysaccharide exposure, lung injury after Libby amphibole asbestos exposure, hyperoxia-induced lung injury, pulmonary hypertension in sickle cell anemia, trauma-hemorrhage, and lung injury post inhalation of Cl₂, bromine or phosgene gases. Physiologically, blood heme concentrations are maintained at low levels by the high binding affinity of serum albumin, hemopexin, and haptoglobin. Hemopexin is a plasma protein with the highest binding affinity to free heme (K_{d} near 10⁻¹³ M). After heme binding, the heme-hemopexin complex is transported to liver and internalized by macrophages through receptor-mediated endocytosis. Therefore, it should be determined, both in vitro and in vivo, whether heme-induced lung inflammation is mediated by SDF-1/CXCR4 dependent leukocyte chemotaxis and activation. It is postulated that heme will increase NRF-1 and CXCR4 expression and downstream signaling.

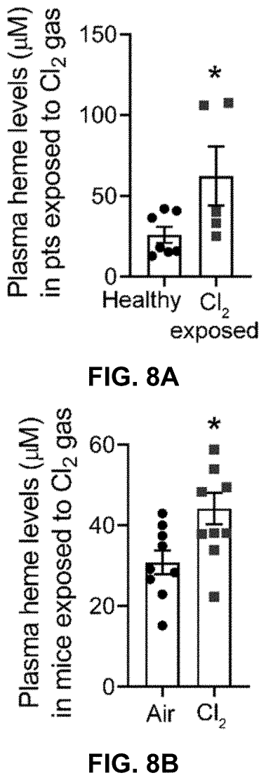

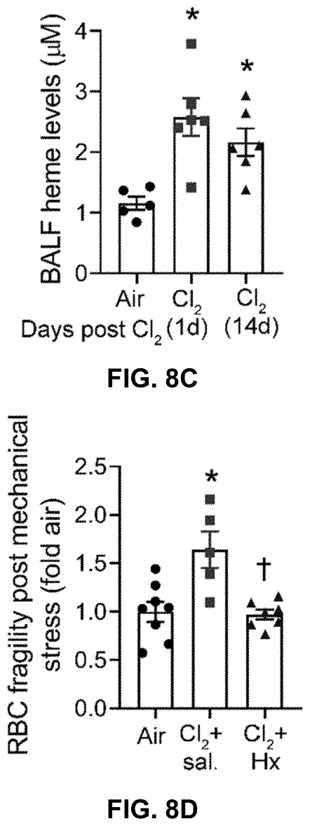

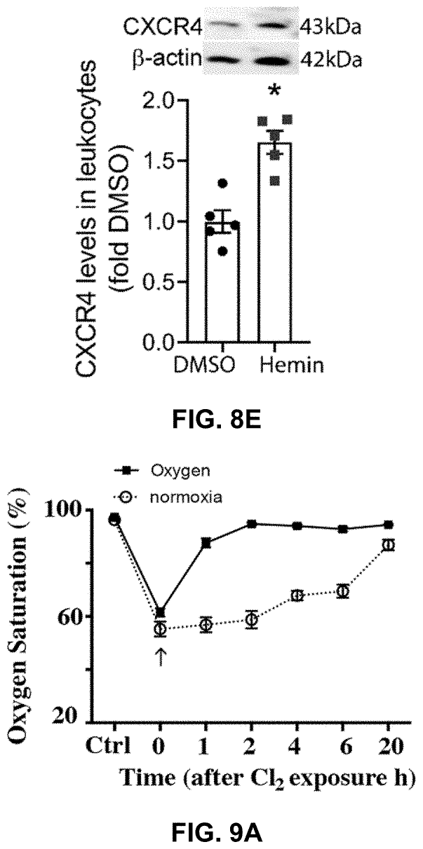

Hemolysis and cell-free heme in humans and animals exposed to Cl₂. It has previously been demonstrated that in comparison to age, sex, and race matched healthy individuals, plasma levels of cell-free heme were elevated in 5 adult humans, 2 days after they were admitted to the University of Alabama at Birmingham Emergency Department, post accidental exposure to Cl₂ gas at the Birmingham water treatment plant (FIG. 8A). Similarly, it has been shown that exposure of adult C57BL/6 male mice to Cl₂ gas (400 ppm, 30 min) increased plasma levels of cell-free heme (24 hours post exposure) (FIG. 8B). It has also been found that cell-free heme levels were also elevated in the BALF of C57BL/6 male mice at 1 and 14 days post exposure to Cl₂ (500 ppm, 30 min) (FIG. 8C), suggesting that the role of cell-free heme in Cl₂ toxicity may persist, long after the source of initial insult is gone. In a previously published study, it was found that the administration of a single injection of the heme scavenging protein, hemopexin (4 mg/g body weight), 1 hour post Cl₂ exposure attenuated hemolysis and reduced plasma levels of cell-free heme (FIG. 8D). Therefore, to begin to determine the role of heme in increasing CXCR4 expression, in a preliminary study, exposed human THP-1 cells (monocyte/macrophages) were exposed to 25 μM of heme (hemin) and measured CXCR4 expression 24 hours later by immunoblotting. The CXCR4 levels were significantly elevated in hemin exposed cells (FIG. 8E) suggesting that heme scavenging in mice post Cl₂ exposure may lower CXCR4 expression and provide a mechanistic insight into the regulation of SDF-1/CXCR4 axis in Cl₂ toxicity.

In vitro studies. To determine the role of cell-free heme in SDF-1/CXCR4 signaling, THP-1 cells (macrophages are the most abundant immune cells in lung) were exposed to hemin (25-50 μM) and the expression of NRF-1 and CXCR4 was measured by immunoblotting at different time intervals post exposure (6-24 hours). Then, it was determined whether the genetic silencing of NRF-1 by siRNA prevents the increase of CXCR4 expression in these cells treated with hemin. It was also determined whether the hemin treated THP-1 cells had increased activity and the ability to migrate through a 5 μm pore diameter filter towards varying concentrations of SDF-1.

In vivo studies. Adult mice (both sexes) were exposed to Cl₂ gas (500 ppm, 30 min) as previously described. Control mice were exposed to room air in the same experimental conditions as Cl₂ exposed mice. Following exposure, mice were returned to room air and then 1 hour later, mice were treated with an intramuscular injection of either saline or purified human hemopexin (4 mg/kg body weight, dissolved in saline). Leukocyte count in BALF and the expression of NRF-1, HIF-1, and CXCR4 in isolated leukocytes and whole lung tissue were assessed by immunoblotting 24 hours post Cl₂ exposure. In addition, the ability of isolated neutrophils to migrate, their activity, and survival was assessed.

Role of hypoxia in regulating SDF-1/CXCR4 signaling post C12 exposure. In recent publications, it was demonstrated that exposure to Cl₂ gas (500 ppm, 30 min) resulted in decreased oxygen saturations in rats for up to 20 hours. Hypoxia-induced HIF-1 plays a significant role in inflammation. The overexpression of HIF-1 in vivo resulted in increased localized inflammation, while loss of the HIF-1 gene decreased the ability of myeloid cells (precursors to leukocytes) to aggregate and migrate to injured tissues. In addition, HIF-1a is involved in the differentiation of myeloid cells into monocytes and macrophages. Hypoxia has also been shown to enhance CXCR4 expression by activating HIF-1 in monocytes, monocyte-derived macrophages, tumor-associated macrophages, endothelial cells, and cancer cells, which is paralleled by increased chemotactic responsiveness to SDF-1. Therefore, the significance of this aim is that it will determine whether hypoxia mediates SDF-1/CXCR4-dependent lung inflammation by leukocytes post C2 exposure. It was postulated that reducing hypoxia would lower SDF-1/CXCR4 signaling and mitigate leukocyte infiltration and activation in C2 exposed mice.

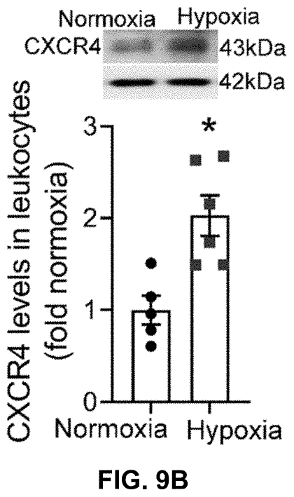

Hypoxia increases CXCR4 expression in human leukocytes. FIG. 9A shows previously published data, where SD rats exposed to C2 gas (500 ppm, 30 min) had a significant decline in oxygen saturation for 20 hours as measured by pulse oximetry. The oxygen saturation was restored to more than 95% in a subset of rats that were placed in 40% oxygen containing environment from where they were removed one at a time to perform pulse oximetry. This experiment was repeated in mice and it was determined whether restoring oxygen saturation attenuates SDF-1/CXCR4 signaling in lungs. FIG. 9B shows elevated expression of CXCR4 in THP-1 cells exposed to 3% oxygen for 24 hours compared to cells housed in normoxic (20% oxygen) conditions suggesting that hypoxia may play a role in leukocyte chemotaxis post C2 exposure.

In vitro studies. To determine the role of hypoxia in SDF-1/CXCR4 signaling, THP-1 cells were exposed to either 20% (normoxic) or 3% (hypoxic) oxygen for 24 hours and the expression of NRF-1 and CXCR4 were measured by immunoblotting at different time intervals post exposure (6-24 hours). Then it was determined whether the genetic silencing of HIF-1 by siRNA prevents the increase of CXCR4 expression in cells exposed to 3% oxygen. It was also determined whether these cells have increased activity and the ability to migrate through a 5 μm pore diameter filter towards varying concentrations of SDF-1 as mentioned previously.

In vivo studies. Adult mice (25-30 g, both sexes) were exposed to Cl₂ gas (500 ppm, 30 min) and then returned to room air and monitored continuously up to 6 hours thereafter at 24 hours. Pulse oximetry was performed using a MouseOx system. In a group of animals, normal oxygen saturations (as measured by pulse oximetry) was restored by housing mice in 40% oxygen containing chambers after Cl₂ exposure where they were continuously monitored up to 24 hours. Blood gas analysis was performed at the end of treatment. Leukocyte count in BALF and the expression of NRF-1, HIF-1, and CXCR4 in isolated leukocytes and whole lung tissue was assessed by immunoblotting 24 hours post Cl₂ exposure. In addition, the ability of isolated neutrophils to migrate, their activity, and survival was assessed as mentioned previously.

In preliminary data (FIG. 8B), there was a mean difference of 13.33 in plasma heme levels between mice exposed to air vs. Cl₂ with a pooled standard deviation of 10.748280. Assuming a 5% significance level and a sample size of 22 (11 air, 11 Cl₂), a two-sample t-test has 80% power to detect a significant difference in plasma heme levels between these 2 groups. Power for future experiments will be calculated based on descriptive statistics. Statistical significance was determined by either two-sample t-test (for 2 groups) or one-way ANOVA (for 3 or more groups) with Tukey's post-test. Depending on whether groups have equal variances, some data was transformed logarithmically. Total mice needed=320.

It is anticipated that THP-1 cells exposed to heme or hypoxia will have elevated levels of NRF-1 and HIF-1, respectively. Both insults enhance CXCR4 expression in cells and increase the migration of THP-1 cells upon SDF-1 stimulation. In vivo, mice treated with hemopexin or supplemental oxygen are expected to have reduced leukocyte infiltration in lungs post Cl₂ exposure. These leukocytes also exhibit reduced CXCR4 expression and attenuated activity and chemotactic capabilities.

Interpretation. These findings suggest that hemolysis and hypoxia are major drivers of SDF-1/CXCR4 mediated inflammation after Cl₂ exposure. The therapies aimed at reducing cell-free heme load or normalizing oxygen saturation immediately post Cl₂ exposure may reduce lung inflammation and mortality. Additionally, FDA-approved HIF-1 antagonists may also have therapeutic utility in mitigating Cl₂ toxicity. In a small set of animals, it was determined whether co-administration of AMD3100 with hemopexin, HIF-1 inhibitor, or oxygen supplementation had additive or synergistic effect in reducing lung inflammation after Cl₂ exposure.

Furthermore, several signaling molecules have been shown to increase the transcription of CXCR4, such as elevated intracellular calcium, cyclic AMP58, cytokines like IL-2, IL-4, IL-7, IL-10, IL-15, and growth factors like TGF-1p, bFGF, VEGF, and EGF. In a previous analysis of 23 cytokines in lungs, none of the above mentioned cytokines were found to be increased post Cl₂ exposure in mice. However, VEGF and TGF-1βlevels have been reported to be increased in the BALF of Cl₂ exposed mice. Therefore, as an alternative strategy, it was determined whether the inhibition of VEGF with an FDA approved drug, bevacizumab, or the inhibition of TGF-1p3 with neutralizing antibody, would inhibit the induction of CXCR4 post Cl₂ exposure.

### Example 5: Intranasal Administration of AMD3100 Attenuates Cl₂-Induced Acute Lung Injury

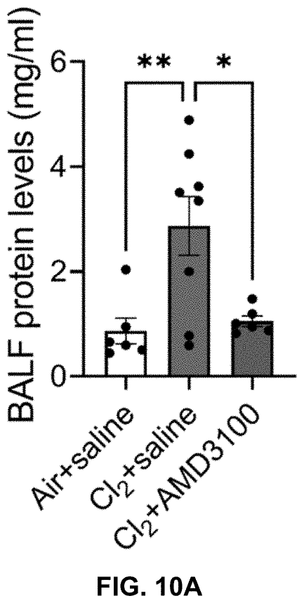

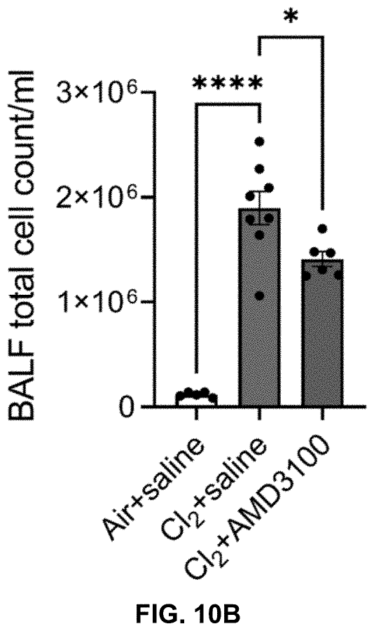

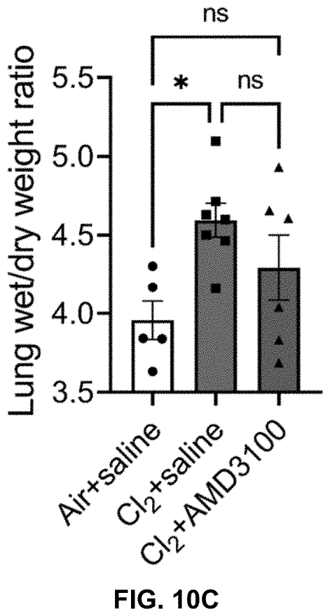

Adult male C57BL/6 mice were exposed to air or Cl₂ gas (500 ppm, 15 min). Several Cl₂ exposed animals received saline, while others received the CXCR4 antagonist, AMD3100 (0.1 mg/kg body weight), 1 hour post exposure. Air exposed mice received saline. One day later, the analysis of BALF demonstrated elevated protein content (sign of endothelial barrier dysfunction and lung injury) (FIG. 10A), increased accumulation of inflammatory cells (sign of lung inflammation) (FIG. 10B) of Cl₂ exposed animals that received saline but not in animals that were given AMD3100. In another subset of animals, the lung wet/dry weight ratio was elevated (sign of lung edema) post Cl₂ exposure in saline treated animals but not in AMD3100 treated mice (FIG. 1)C). Taken together, these results indicate that intranasal administration of AMD3100 is an effective therapeutic strategy against Cl₂-induced acute lung injury.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Abraham E, et al. Neutrophils as early immunologic effectors in hemorrhage- or endotoxemia-induced acute lung injury. American journal of physiology Lung cellular and molecular physiology 2000; 279:L1137-45.
2. Aggarwal S, et al. Heme attenuates beta-endorphin levels in leukocytes of HIV positive individuals with chronic widespread pain. Redox biology 2020; 36:101684.
3. Aggarwal S, et al. Heme Attenuation Ameliorates Irritant Gas Inhalation-Induced Acute Lung Injury. Antioxidants & redox signaling 2016; 24:99-112.
4. Aggarwal S, et al. Heme scavenging reduces pulmonary endoplasmic reticulum stress, fibrosis, and emphysema. JCI insight 2018; 3.
5. Aggarwal S, et al. Phosgene inhalation causes hemolysis and acute lung injury. Toxicology letters 2019; 312:204-13.
6. Aggarwal S, et al. Reactive species generated by heme impair alveolar epithelial sodium channel function in acute respiratory distress syndrome. Redox biology 2020; 36:101592.
7. Aggarwal S A, et al. Heme Scavenging Reduces Pulmonary Endoplasmic Reticulum Stress, Fibrosis, and Emphysema. JCI insight 2018; 3:e120694.
8. Ahmad S, et al. Sarcoendoplasmic reticulum Ca(2+) ATPase. A critical target in chlorine inhalation-induced cardiotoxicity. American journal of respiratory cell and molecular biology 2015; 52:492-502.
9. Balla J, et al. Heme, heme oxygenase, and ferritin: how the vascular endothelium survives (and dies) in an iron-rich environment. Antioxidants & redox signaling 2007; 9:2119-37.
10. Belperio J A, et al. Critical role for CXCR2 and CXCR2 ligands during the pathogenesis of ventilator-induced lung injury. The Journal of clinical investigation 2002; 110:1703-16.
11. Bhatia M, et al. Treatment with neutralising antibody against cytokine induced neutrophil chemoattractant (CINC) protects rats against acute pancreatitis associated lung injury. Gut 2000; 47:838-44.
12. Bleul C C, et al. The lymphocyte chemoattractant SDF-1 is a ligand for LESTR/fusin and blocks HIV-1 entry. Nature 1996; 382:829-33.
13. Bradfield P F, et al. Rheumatoid fibroblast-like synoviocytes overexpress the chemokine stromal cell-derived factor 1 (CXCL12), which supports distinct patterns and rates of CD4+ and CD8+ T cell migration within synovial tissue. Arthritis and rheumatism 2003; 48:2472-82.
14. Carlisle M, et al. Chlorine-induced cardiopulmonary injury. Annals of the New York Academy of Sciences 2016; 1374:159-67.
15. Chang W, et al. Inhibition of chlorine-induced lung injury by the type 4 phosphodiesterase inhibitor rolipram. Toxicology and applied pharmacology 2012; 263:251-8.
16. Cheng X, et al. The Role of SDF-1/CXCR4/CXCR7 in Neuronal Regeneration after Cerebral Ischemia. Frontiers in neuroscience 2017; 11:590.
17. Colletti L M, et al. Chemokine expression during hepatic ischemia/reperfusion-induced lung injury in the rat. The role of epithelial neutrophil activating protein. The Journal of clinical investigation 1995; 95:134-41.
18. Cramer T, et al. HIF-1alpha is essential for myeloid cell-mediated inflammation. Cell 2003; 112:645-57.
19. Cristillo A D, et al. Up-regulation of HIV coreceptor CXCR4 expression in human T lymphocytes is mediated in part by a cAMP-responsive element. FASEB journal: official publication of the Federation of American Societies for Experimental Biology 2002; 16:354-64.
20. Cully M. Lung disease: CXCR7 activation overrides lung fibrosis. Nature reviews Drug discovery 2016; 15:160.
21. De Clercq E. Mozobil(R) (Plerixafor, AMD3100), 10 years after its approval by the US Food and Drug Administration. Antiviral chemistry & chemotherapy 2019; 27:2040206619829382.
22. De Falco E, et al. SDF-1 involvement in endothelial phenotype and ischemia-induced recruitment of bone marrow progenitor cells. Blood 2004; 104:3472-82.
23. Dennery P A, et al. Resistance to hyperoxia with heme oxygenase-1 disruption: role of iron. Free radical biology & medicine 2003; 34:124-33.
24. Domanska U M, et al. A review on CXCR4/CXCL12 axis in oncology: no place to hide. European journal of cancer 2013; 49:219-30.
25. Donnelly S C, et al. Interleukin-8 and development of adult respiratory distress syndrome in at-risk patient groups. Lancet 1993; 341:643-7.
26. Donzella G A, et al. AMD3100, a small molecule inhibitor of HIV-1 entry via the CXCR4 co-receptor. Nature medicine 1998; 4:72-7.
27. Drummond S, et al. CXCR4 blockade attenuates hyperoxia-induced lung injury in neonatal rats. Neonatology 2015; 107:304-11.
28. Duniho S M, et al. Acute changes in lung histopathology and bronchoalveolar lavage parameters in mice exposed to the choking agent gas phosgene. Toxicologic pathology 2002; 30:339-49.
29. Eash K J, et al. CXCR4 is a key regulator of neutrophil release from the bone marrow under basal and stress granulopoiesis conditions. Blood 2009; 113:4711-9.
30. Eggenhofer E, et al. The life and fate of mesenchymal stem cells. Frontiers in immunology 2014; 5:148.
31. Feil C, et al. Endothelial cells differentially express functional CXC-chemokine receptor-4 (CXCR-4/fusin) under the control of autocrine activity and exogenous cytokines. Biochemical and biophysical research communications 1998; 247:38-45.
32. Folkesson H G, et al. Acid aspiration-induced lung injury in rabbits is mediated by interleukin-8-dependent mechanisms. The Journal of clinical investigation 1995; 96:107-16.
33. Ford D A, et al. Formation of chlorinated lipids post-chlorine gas exposure. Journal of lipid research 2016; 57:1529-40.
34. Fortunati E, et al. Human neutrophils switch to an activated phenotype after homing to the lung irrespective of inflammatory disease. Clinical and experimental immunology 2009; 155:559-66.
35. Gessner M A, et al. Chlorine gas exposure increases susceptibility to invasive lung fungal infection. American journal of physiology Lung cellular and molecular physiology 2013; 304:L765-73.
36. Gonzalo J A, et al. Critical involvement of the chemotactic axis CXCR4/stromal cell-derived factor-1 alpha in the inflammatory component of allergic airway disease. Journal of immunology 2000; 165:499-508.
37. Goodman R B, et al. Inflammatory cytokines in patients with persistence of the acute respiratory distress syndrome. American journal of respiratory and critical care medicine 1996; 154:602-11.
38. Grygorczyk R, et al. Effects of Hypoxia on Erythrocyte Membrane Properties-Implications for Intravascular Hemolysis and Purinergic Control of Blood Flow. Frontiers in physiology 2017; 8:1110.
39. Guan G, et al. The HIF-1alpha/CXCR4 pathway supports hypoxia-induced metastasis of human osteosarcoma cells. Cancer letters 2015; 357:254-64.
40. Gummin D D, et al. 2016 Annual Report of the American Association of Poison Control Centers' National Poison Data System (NPDS): 34th Annual Report. Clinical toxicology 2017; 55:1072-252.
41. Hamamoto Y, et al. Montelukast reduces inhaled chlorine triggered airway hyperresponsiveness and airway inflammation in the mouse. British journal of pharmacology 2017; 174:3346-58.
42. Hashimoto N, et al. Bone marrow-derived progenitor cells in pulmonary fibrosis. The Journal of clinical investigation 2004; 113:243-52.
43. Heide K, et al. On the Heme-Binding Capacity of Hemopexin. Clin Chim Acta 1964; 10:460-9.
44. Hendrix C W, et al. Pharmacokinetics and safety of AMD-3100, a novel antagonist of the CXCR-4 chemokine receptor, in human volunteers. Antimicrobial agents and chemotherapy 2000; 44:1667-73.
45. Honavar J, et al. Chlorine gas exposure causes systemic endothelial dysfunction by inhibiting endothelial nitric oxide synthase-dependent signaling. American journal of respiratory cell and molecular biology 2011; 45:419-25.
46. Honavar J, et al. Chlorine gas exposure disrupts nitric oxide homeostasis in the pulmonary vasculature. Toxicology 2014; 321:96-102.
47. Honavar J, et al. Nitrite therapy improves survival postexposure to chlorine gas. American journal of physiology Lung cellular and molecular physiology 2014; 307:L888-94.
48. Honavar J, et al. Nitrite therapy prevents chlorine gas toxicity in rabbits. Toxicology letters 2017; 271:20-5.
49. Hvidberg V, et al. Identification of the receptor scavenging hemopexin-heme complexes. Blood 2005; 106:2572-9.
50. Ishikawa T, et al. Hypoxia enhances CXCR4 expression by activating HIF-1 in oral squamous cell carcinoma. Oncology reports 2009; 21:707-12.
51. Janssens R, et al. The unique structural and functional features of CXCL12. Cellular & molecular immunology 2018; 15:299-311.
52. Jourdan P, et al. Cytokines and cell surface molecules independently induce CXCR4 expression on CD4+ CCR7+ human memory T cells. Journal of immunology 2000; 165:716-24.
53. Jurkuvenaite A, et al. Upregulation of autophagy decreases chlorine-induced mitochondrial injury and lung inflammation. Free radical biology & medicine 2015; 85:83-94.
54. Kato G J, et al. Intravascular hemolysis and the pathophysiology of sickle cell disease. The Journal of clinical investigation 2017; 127:750-60.
55. Killander J. Separation of Human Heme- and Hemoglobin-Binding Plasma Proteins, Ceruloplasmin and Albumin by Gel Filtration. Biochim Biophys Acta 1964; 93:1-14.
56. Konrad F M, et al. Inhibition of SDF-1 receptors CXCR4 and CXCR7 attenuates acute pulmonary inflammation via the adenosine A2B-receptor on blood cells. Cell death & disease 2017; 8:e2832.
57. Kose A, et al. Myocardial infarction, acute ischemic stroke, and hyperglycemia triggered by acute chlorine gas inhalation. The American journal of emergency medicine 2009; 27:1022 e1-4.
58. Kruizinga R C, et al. Role of chemokines and their receptors in cancer. Current pharmaceutical design 2009; 15:3396-416.
59. Kryczek I, et al. CXCL12 and vascular endothelial growth factor synergistically induce neoangiogenesis in human ovarian cancers. Cancer research 2005; 65:465-72.
60. Kubo H, et al. Complement fragment-induced release of neutrophils from bone marrow and sequestration within pulmonary capillaries in rabbits. Blood 1998; 92:283-90.
61. Kubo H, et al. Preservation of complement-induced lung injury in mice with deficiency of NADPH oxidase. The Journal of clinical investigation 1996; 97:2680-4.
62. Kuhns D B, et al. Isolation and Functional Analysis of Human Neutrophils. Curr Protoc Immunol 2015; 111:723 1-7 16.
63. Lam A, et al. Role of heme in bromine-induced lung injury. Annals of the New York Academy of Sciences 2016; 1374:105-10.
64. Lazrak A, et al. Hyaluronan and halogen-induced airway hyperresponsiveness and lung injury. Annals of the New York Academy of Sciences 2020.
65. Lazrak A, et al. Hyaluronan mediates airway hyperresponsiveness in oxidative lung injury. American journal of physiology Lung cellular and molecular physiology 2015; 308:L891-903.
66. Li C, et al. Chlorine induces the unfolded protein response in murine lungs and skin. American journal of respiratory cell and molecular biology 2013; 49:197-203.
67. Li F, et al. The autocrine CXCR4/CXCL12 axis contributes to lung fibrosis through modulation of lung fibroblast activity. Experimental and therapeutic medicine 2020; 19:1844-54.
68. Li L, et al. Prolonged exposure to LPS increases iron, heme, and p22phox levels and NADPH oxidase activity in human aortic endothelial cells: inhibition by desferrioxamine. Arterioscler Thromb Vasc Biol 2009; 29:732-8.
69. Li W, et al. Phosgene-induced acute lung injury (ALI): differences from chlorine-induced ALI and attempts to translate toxicology to clinical medicine. Clinical and translational medicine 2017; 6:19.
70. Liles W C, et al. Mobilization of hematopoietic progenitor cells in healthy volunteers by AMD3100, a CXCR4 antagonist. Blood 2003; 102:2728-30.
71. Lukacs N W, et al. AMD3100, a CxCR4 antagonist, attenuates allergic lung inflammation and airway hyperreactivity. The American journal of pathology 2002; 160:1353-60.
72. Ma D M, et al. SDF-1/CXCR7 axis regulates the proliferation, invasion, adhesion, and angiogenesis of gastric cancer cells. World journal of surgical oncology 2016; 14:256.
73. Machogu E M, et al. How I treat hypoxia in adults with hemoglobinopathies and hemolytic disorders. Blood 2018; 132:1770-80.
74. Makino H, et al. Antifibrotic effects of CXCR4 antagonist in bleomycin-induced pulmonary fibrosis in mice. The journal of medical investigation: JMI 2013; 60:127-37.
75. Marchese A, et al. Agonist-promoted ubiquitination of the G protein-coupled receptor CXCR4 mediates lysosomal sorting. The Journal of biological chemistry 2001; 276:45509-12.
76. Marchese A. Ubiquitination of chemokine receptors. Methods in enzymology 2009; 460:413-22.
77. Martin C, et al. Chemokines acting via CXCR2 and CXCR4 control the release of neutrophils from the bone marrow and their return following senescence. Immunity 2003; 19:583-93.
78. Matthay M A, et al. Elevated concentrations of leukotriene D4 in pulmonary edema fluid of patients with the adult respiratory distress syndrome. Journal of clinical immunology 1984; 4:479-83.
79. Matthys P, et al. AMD3100, a potent and specific antagonist of the stromal cell-derived factor-1 chemokine receptor CXCR4, inhibits autoimmune joint inflammation in IFN-gamma receptor-deficient mice. Journal of immunology 2001; 167:4686-92.
80. Matute-Bello G, et al. An official American Thoracic Society workshop report: features and measurements of experimental acute lung injury in animals. American journal of respiratory cell and molecular biology 2011; 44:725-38.
81. McDermott D H, et al. A phase 1 clinical trial of long-term, low-dose treatment of WHIM syndrome with the CXCR4 antagonist plerixafor. Blood 2014; 123:2308-16.
82. Miller E J, et al. Elevated levels of NAP-1/interleukin-8 are present in the airspaces of patients with the adult respiratory distress syndrome and are associated with increased mortality. The American review of respiratory disease 1992; 146:427-32.
83. Miller Y I, et al. Role of hemopexin in protection of low-density lipoprotein against hemoglobin-induced oxidation. Biochemistry 1996; 35:13112-7.
84. Mo Y, et al. Abnormal epithelial structure and chronic lung inflammation after repair of chlorine-induced airway injury. American journal of physiology Lung cellular and molecular physiology 2015; 308:L168-78.
85. Moriuchi M, et al. Cloning and analysis of the promoter region of CXCR4, a coreceptor for HIV-1 entry. Journal of immunology 1997; 159:4322-9.
86. Muller A, et al. Involvement of chemokine receptors in breast cancer metastasis. Nature 2001; 410:50-6.
87. Murakami T, et al. Expression of CXC chemokine receptor-4 enhances the pulmonary metastatic potential of murine B16 melanoma cells. Cancer research 2002; 62:7328-34.
88. Nagasawa T, et al. Defects of B-cell lymphopoiesis and bone-marrow myelopoiesis in mice lacking the CXC chemokine PBSF/SDF-1. Nature 1996; 382:635-8.
89. Nagel R L, et al. The binding of hemoglobin to haptoglobin and its relation to subunit dissociation of hemoglobin. J Biol Chem 1971; 246:69-73.
90. Nanki T, et al. Stromal cell-derived factor-1-CXC chemokine receptor 4 interactions play a central role in CD4+ T cell accumulation in rheumatoid arthritis synovium. Journal of immunology 2000; 165:6590-8.
91. Ngamsri K C, et al. The Pivotal Role of CXCR7 in Stabilization of the Pulmonary Epithelial Barrier in Acute Pulmonary Inflammation. Journal of immunology 2017; 198:2403-13.
92. Oberlin E, et al. The CXC chemokine SDF-1 is the ligand for LESTR/fusin and prevents infection by T-cell-line-adapted HIV-1. Nature 1996; 382:833-5.
93. Oda T, et al. Activation of hypoxia-inducible factor 1 during macrophage differentiation. American journal of physiology Cell physiology 2006; 291:C104-13.
94. Onnis B, et al. Development of HIF-1 inhibitors for cancer therapy. Journal of cellular and molecular medicine 2009; 13:2780-6.
95. Pablos J L, et al. Synoviocyte-derived CXCL12 is displayed on endothelium and induces angiogenesis in rheumatoid arthritis. Journal of immunology 2003; 170:2147-52.
96. Petty J M, et al. Pulmonary stromal-derived factor-1 expression and effect on neutrophil recruitment during acute lung injury. Journal of immunology 2007; 178:8148-57.
97. Phillips R J, et al. Circulating fibrocytes traffic to the lungs in response to CXCL12 and mediate fibrosis. The Journal of clinical investigation 2004; 114:438-46.
98. Phillips R J, et al. Epidermal growth factor and hypoxia-induced expression of CXC chemokine receptor 4 on non-small cell lung cancer cells is regulated by the phosphatidylinositol 3-kinase/PTEN/AKT/mammalian target of rapamycin signaling pathway and activation of hypoxia inducible factor-1alpha. The Journal of biological chemistry 2005; 280:22473-81.
99. Phillips R J, et al. The stromal derived factor-1/CXCL12-CXC chemokine receptor 4 biological axis in non-small cell lung cancer metastases. American journal of respiratory and critical care medicine 2003; 167:1676-86.
100. Rajasekaran D, et al. Macrophage Migration Inhibitory Factor-CXCR4 Receptor Interactions: EVIDENCE FOR PARTIAL ALLOSTERIC AGONISM IN COMPARISON WITH CXCL12 CHEMOKINE. The Journal of biological chemistry 2016; 291:15881-95.
101. Saini V, et al. CXC chemokine receptor 4 is a cell surface receptor for extracellular ubiquitin. The Journal of biological chemistry 2010; 285:15566-76.
102. Salcedo R, et al. Vascular endothelial growth factor and basic fibroblast growth factor induce expression of CXCR4 on human endothelial cells: In vivo neovascularization induced by stromal-derived factor-1alpha. The American journal of pathology 1999; 154:1125-35.
103. Santiago B, et al. CXCL12 gene expression is upregulated by hypoxia and growth arrest but not by inflammatory cytokines in rheumatoid synovial fibroblasts. Cytokine 2011; 53:184-90.
104. Schioppa T, et al. Regulation of the chemokine receptor CXCR4 by hypoxia. The Journal of experimental medicine 2003; 198:1391-402.
105. Sciuto A M, et al. Disruption of gas exchange in mice after exposure to the chemical threat agent phosgene. Military medicine 2001; 166:809-14.
106. Shannahan J H, et al. The role of iron in Libby amphibole-induced acute lung injury and inflammation. Inhal Toxicol 2011; 23:313-23.
107. Shao Y, et al. Overexpression of CXCR7 promotes mesenchymal stem cells to repair phosgene-induced acute lung injury in rats. Biomedicine & pharmacotherapy=Biomedecine & pharmacotherapie 2019; 109:1233-9.
108. Sharafkhaneh A, et al. Pathogenesis of emphysema: from the bench to the bedside.

Proceedings of the American Thoracic Society 2008; 5:475-7.
109. Shi H, et al. Effects of SDF-1/CXCR4 on Acute Lung Injury Induced by Cardiopulmonary Bypass. Inflammation 2017; 40:937-45.
110. Smith M C, et al. CXCR4 regulates growth of both primary and metastatic breast cancer. Cancer research 2004; 64:8604-12.
111. Sohni A, et al. Mesenchymal stem cells migration homing and tracking. Stem cells international 2013; 2013:130763.
112. Song J S, et al. Inhibitory effect of CXC chemokine receptor 4 antagonist AMD3100 on bleomycin induced murine pulmonary fibrosis. Experimental & molecular medicine 2010; 42:465-72.
113. Song W, et al. Postexposure administration of a {beta}2-agonist decreases chlorine-induced airway hyperreactivity in mice. American journal of respiratory cell and molecular biology 2011; 45:88-94.
114. Stapley R, et al. Red blood cell washing, nitrite therapy, and antiheme therapies prevent stored red blood cell toxicity after trauma-hemorrhage. Free radical biology & medicine 2015; 85:207-18.
115. Steinberg K P, et al. Evolution of bronchoalveolar cell populations in the adult respiratory distress syndrome. American journal of respiratory and critical care medicine 1994; 150:113-22.
116. Struyf S, et al. Chemokines synergize in the recruitment of circulating neutrophils into inflamed tissue. European journal of immunology 2005; 35:1583-91.
117. Sue R D, et al. CXCR2 is critical to hyperoxia-induced lung injury. Journal of immunology 2004; 172:3860-8.
118. Suratt B T, et al. Role of the CXCR4/SDF-1 chemokine axis in circulating neutrophil homeostasis. Blood 2004; 104:565-71.
119. Tamamura H, et al. Enhancement of the T140-based pharmacophores leads to the development of more potent and bio-stable CXCR4 antagonists. Organic & biomolecular chemistry 2003; 1:3663-9.
120. Teicher B A, et al. CXCL12 (SDF-1)/CXCR4 pathway in cancer. Clinical cancer research: an official journal of the American Association for Cancer Research 2010; 16:2927-31.
121. Tsai P K, et al. Elevated plasma stromal-cell-derived factor-1 protein levels correlate with severity in patients with community-acquired pneumonia. Disease markers 2014; 2014:829706.
122. Van Sickle D, et al. Acute health effects after exposure to chlorine gas released after a train derailment. The American journal of emergency medicine 2009; 27:1-7.
123. Wang J, et al. Role of tyrosine phosphorylation in ligand-independent sequestration of CXCR4 in human primary monocytes-macrophages. The Journal of biological chemistry 2001; 276:49236-43.
124. Wegner S A, et al. Genomic organization and functional characterization of the chemokine receptor CXCR4, a major entry co-receptor for human immunodeficiency virus type 1. The Journal of biological chemistry 1998; 273:4754-60.
125. White C W, et al. Chlorine gas inhalation: human clinical evidence of toxicity and experience in animal models. Proceedings of the American Thoracic Society 2010; 7:257-63.
126. Wigenstam E, et al. Acute respiratory changes and pulmonary inflammation involving a pathway of TGF-beta1 induction in a rat model of chlorine-induced lung injury. Toxicology and applied pharmacology 2016; 309:44-54.
127. Xu J, et al. Role of the SDF-1/CXCR4 axis in the pathogenesis of lung injury and fibrosis. American journal of respiratory cell and molecular biology 2007; 37:291-9.
128. Yamada M, et al. The increase in surface CXCR4 expression on lung extravascular neutrophils and its effects on neutrophils during endotoxin-induced lung injury. Cellular & molecular immunology 2011; 8:305-14.
129. Zaky A, et al. Chlorine inhalation-induced myocardial depression and failure. Physiological reports 2015; 3.

What is claimed is:

1. A method for treating or preventing at least one symptom of exposure to a toxic gas in a subject, the method comprising administering a composition comprising AMD3100 or a pharmaceutically acceptable salt thereof to the subject.

2. The method of claim 1, wherein the toxic gas comprises chlorine, bromine, phosgene, or any combination thereof.

3. The method of claim 2, wherein the toxic gas is chlorine.

4. The method of claim 1, wherein the at least one symptom comprises difficulty breathing, cough, lung inflammation, hemolysis, increased plasma levels of cell-free heme, remodeling in large or small airways, obstruction in large or small airways, fibrosis, damage to alveolar septa, hypoxia, lung edema, protein extravasation into lungs, another decrease in lung function, or any combination thereof.

5. The method of claim 1, wherein the subject is a human.

6. The method of claim 1, wherein the composition is administered by intermuscular injection or inhalation.

7. The method of claim 1, wherein the composition is administered from one hour to 7 days following exposure.

8. The method of claim 1, wherein the composition is administered in an amount comprising from about 0.01 to about 0.25 mg of AMD3100 per kg of subject body weight.

9. The method of claim 1, wherein the pharmaceutically acceptable salt of AMD3100 comprises a hydrochloride salt.

10. The method of claim 1, further comprising administering at least one additional treatment to the subject.

11. The method of claim 13, wherein the at least one additional treatment comprises administering an HIF-1 inhibitor, hemopexin, a VEGF inhibitor, a TGF-1βinhibitor, supplemental oxygen, or any combination thereof.

12. The method of claim 14, wherein the HIF-1 inhibitor comprises topotecan, digoxin, digitoxin, temsirolimus, everolimus, bortezomib, or any combination thereof.

13. The method of claim 1, wherein performing the method reduces a level of at least one of inflammatory cells, cell-free heme, CXCR4, or SDF-1 in the subject in plasma, blood, bronchoalveolar lavage fluid, or any combination thereof, compared to an untreated subject exposed to the toxic gas.

14. A composition for treating or preventing at least one symptom of exposure to a toxic gas in a subject, the composition comprising AMD3100 or a pharmaceutically acceptable salt thereof to the subject.

15. The composition of claim 14, wherein the at least one symptom comprises difficulty breathing, cough, lung inflammation, hemolysis, increased plasma levels of cell-free heme, remodeling in large or small airways, obstruction in large or small airways, fibrosis, damage to alveolar septa, hypoxia, lung edema, protein extravasation into lungs, another decrease in lung function, or any combination thereof.

16. The composition of claim 14, wherein the composition reduces at least one of inflammatory cells, cell-free heme, CXCR4, or SDF-1 in the blood, plasma, bronchoalveolar lavage fluid, or any combination thereof.

17. The composition of claim 14, wherein the pharmaceutically acceptable salt of AMD3100 comprises a hydrochloride salt.

18. The composition of claim 14, further comprising at least one additional therapeutic agent.

19. The composition of claim 18, wherein the at least one additional therapeutic agent comprises an HIF-1 inhibitor, hemopexin, a VEGF inhibitor, a TGF-1βinhibitor, or any combination thereof.

20. The method of claim 19, wherein the HIF-1 inhibitor comprises topotecan, digoxin, digitoxin, temsirolimus, everolimus, bortezomib, or any combination thereof.
